(19) United States
(12) Patent Application Publication
(10) Pub. No.: US 2022/0160223 A1
(43) Pub. Date: May 26, 2022

(54) Methods and Systems for Evaluating Vision Acuity and/or Conducting Visual Field Tests in a Head-Mounted Vision Device

(71) Applicant: Irisvision, Inc., Pleasanton, CA (US)

(72) Inventors: Christopher Kent Bradley, Baltimore, MD (US); Dino De Cicco, Pleasanton, CA (US)

(21) Appl. No.: 17/456,490

(22) Filed: Nov. 24, 2021

Related U.S. Application Data
(60) Provisional application No. 63/223,005, filed on Jul. 18, 2021.
(60) Provisional application No. 63/118,539, filed on Nov. 25, 2020.

(51) Int. Cl. A61B 3/00; A61B 3/024; A61B 3/032; G02B 27/01

(52) U.S. Cl. CPC A61B 3/005; G02B 27/0176; A61B 3/032; A61B 3/024

(57) ABSTRACT

Systems and methods for diagnosing a user's vision condition by combining results of one or more vision tests taken by the user via a head-mounted vision device (HMVD) are provided. A user is provided with a first vision test and a second vision test via the HMVD and takes each test a predefined number of times and in a predefined order. Results are obtained from each test taken by the user, combined to obtain a combined test result for the user, and then used to diagnose the user's vision condition.

### CROSS-REFERENCE

The present application relies on U.S. Patent Provisional Application No. 63/223,005, titled “Methods and Systems for Determining a Variety of Vision Parameters in a Head-Mounted Vision Device” and filed on Jul. 18, 2021, for priority.

The present application also relies on U.S. Patent Provisional Application No. 63/118,538, titled “Method and System for Determining Vision Acuity in a Head-Mounted Vision Device” and filed on Nov. 25, 2020, for priority.

The above-mentioned applications are herein incorporated by reference in their entirety.

### FIELD

The present specification relates to vision assist and/or diagnostic systems and methods. Specifically, the embodiments disclosed herein describe head-mounted systems with software configured to accurately monitor, quantify, and determine a plurality of parameters representative of the vision of users of the head-mounted systems. More specifically, the embodiments disclosed herein describe head-mounted systems with software configured to accurately monitor, quantify, and determine the vision acuity and/or visual field of users of the head-mounted systems.

### BACKGROUND

Head-mounted devices typically comprise a video camera, a processor, and a display, which are integrated into a head-mounted housing worn by the user. In some embodiments, a head-mounted device may be configured to process, optically modify, or capture images of the environment and subject those images to specialized processing in order to diagnose and/or account for deficiencies in the user's eyesight. In embodiments, different modes of operating the device are provided that enable the user to configure the device for specific applications. In some embodiments, the mode of operation includes a diagnostic mode in which a wearer's degree of visual acuity may be assessed. In particular, the head-mounted visual assist device may be configured to execute a plurality of visual tests, elicit responses from the wearer which are input into the device, and, based on those responses, determine a value indicative of the wearer's visual acuity.

Conventional visual acuity tests are eye exams that determine how well an individual can see a letter or symbol of a given size from a predefined distance. For example, in a Snellen test, letters are arranged in rows and/or columns. From row to row, the letters have different sizes, typically decreasing in size as one visually progresses from a higher row to a lower row. Standing 14 to 20 feet away, an individual attempts to identify each letter accurately. A value representative of the individual's visual acuity may be determined based on how far the individual is able to accurately progress in the chart (the smallest letter the individual is able to accurately identify). Alternatively, in a Random or Tumbling E test, an individual is presented a series of “E” images of different sizes and instructed to identify the direction each letter “E” is facing, such as up, down, left, or right. Again, a value representative of the individual's visual acuity may be determined based on how far the individual is able to accurately progress in the chart (the smallest letter the individual is able to accurately identify). Regardless of the type of visual acuity test being used, a conventional visual acuity test requires an individual to accurately identify the type of, or orientation of, differently sized letters, symbols, or figures, generally referred to as optotypes.

While conventional visual acuity tests may be effectuated in a head-mounted visual assist or diagnostic device by performing a similar display of optotypes, such tests are fundamentally limited by the proximity of the display to the individual's eyes. Specifically, the size decrease of optotypes is limited by the pixel size of the display in angular relation to the individual's eyes. Therefore, it becomes difficult to accurately assess the visual acuity of an individual who may have a visual acuity of 20/80 or better (depending upon the resolution of a display shown to the individual) in a head-mounted visual assist or diagnostic device using a display with a conventional level of resolution. More specifically, assessing the visual acuity of an individual in a head-mounted visual assist or diagnostic device is limited by the resolution of the display since determining higher levels of visual acuity require presenting smaller and smaller optotypes for which, assuming a conventional pixel resolution and a magnifying lens in the head-mounted device, important structural details, such as gaps, get lost, are not distinctly presented, or are otherwise blurred.

Assessing the visual acuity of an individual outside of a conventional clinic, such as at home, is also limited by environmental factors. Tests that are performed without the guidance of a clinician and use a distance of several feet to the optotypes are difficult to administer consistently because users find it difficult to accurately and consistently set the right distance between themselves and the displayed optotypes and because users are unable to establish the right degree of ambient light required for a given test. Such inconsistencies can result in substantial variations in, and inaccuracies in, measurements, diagnoses and treatments.

Conventionally, testing a user's visual field and/or peripheral vision is performed with dedicated devices and systems. Two common vision tests are the Humphrey visual field test (VFT) and the Amsler grid test. In VFT, a user is provided with a device that displays visual stimuli (typically a small spot of light) at a predetermined set of test locations in the user's peripheral visual field. The user is instructed to fixate at the center of the visual field and to detect any spot of light that appears in the peripheral visual field. Typically, the spot of light is presented at each test location multiple times, and at different contrast levels. The contrast level at which the probability of detection is at a predetermined level (usually set to 50% probability) represents the contrast threshold at that test location. If the contrast threshold is sufficiently high (equivalently, contrast sensitivity is sufficiently low), the system determines that the user may have a deficit in his or her visual field at that test location. Stimulus presentation at any test location may be optimized to reduce the number of measurements by leveraging estimates of contrast thresholds at neighboring test locations, dynamically changing the time between stimulus presentations based on the distribution of user response times and leveraging normative databases of contrast sensitivity for different age groups. Examples of algorithms that implement such optimization procedures are SITA, ZEST and SWeLZ. In certain cases, the VFT may be used to present the stimulus at only the highest possible contrast level as a fast screening method.

The Amsler Grid test is a tool for discovering a user's visual impairments and entails a user looking at a printed grid of squares to determine whether there are any perceived artifacts or irregularities in the grid. The grid structure used for the test is simple and the contents within the grid are usually known to the user, which makes it easier to observe and note any differences between the actual content and what is visually perceived. Describing abnormal vision can be done on paper with a similar grid where a user can mark the areas of distortion for different kinds of distortions that may occur. Amsler grids are commonly 20×20 squares that may be placed at a distance to a user's eye such that each square subtends one degree of visual angle. Different versions of the grid may have varying features, such as color, for example. The Amsler Grid test is subjective; thus, a severity of vision impairment cannot easily be quantified through the test. The test is intended to be a quick and simple screening tool to determine common vision abnormalities such as, but not limited to scotoma, voids, holes, blind spots, and missing area in a user's vison.

Since vision tests such as the VFT and Amsler grid tests are conducted using different systems and settings, the test application and processes vary between the systems and vary between physicians applying the tests and vary over time, making the test results less comparable and less precise due to introduction of these multiple variations. There is thus a need for a controlled environment for conducting these vision tests, such that the influence of the variations is minimized. Additionally, the outputs of the vision tests are typically stored in paper format or in a digital format in a manner that fails to aggregate, integrate, or cross-correlate the results of the tests, making comparisons between tests difficult and impractical.

Accordingly, there is need for a controlled environment for vision testing which has reduced variance and provides improved diagnostic results allowing for comparison between users and over time. There is also a need for a method of measuring visual acuity of a user and enhancing said measured visual acuity while at the same time compensating for the resolution limitations of the head mounted display. Furthermore, there is a need for being able to accurately assess the visual acuity of an individual who may have a visual acuity of 20/80 or better in a head-mounted visual assist device. Finally, and in general, there is a need for an improved controlled vision assessment system where key environmental conditions are effectively controlled and monitored to insure decreased variations in, and increased accuracies of, measurements, diagnoses and treatments.

### SUMMARY

The following embodiments and aspects thereof are described and illustrated in conjunction with systems, tools and methods, which are meant to be exemplary and illustrative, not limiting in scope. The present application discloses numerous embodiments.

The present specification discloses a method of evaluating a user's visual field using a head-mounted device configured to be positioned on the user's head, wherein the device comprises at least one processor, a display in data communication with the at least one processor, and a non-transient memory in data communication with the at least one processor and adapted to store programmatic instructions that, when executed, execute said method, the method comprising: generating a first plurality of visual stimuli, wherein the first plurality of visual stimuli is presented in a form of a grid defined by two or more vertical lines intersecting two or more horizontal lines, wherein the grid covers a first plurality of coordinate locations in the visual field, and wherein each of the first plurality of visual stimuli has at least one of a first plurality of characteristics; causing the first plurality of visual stimuli to be displayed on the display in accordance with its first plurality of characteristics; detecting a discrepancy based on a comparison of the first plurality of characteristics with a user's response that is indicative of the visual characteristics of the first plurality of visual stimuli experienced by the user; storing the detected discrepancy as a first set of data; using the first set of data to generate a second plurality of visual stimuli, wherein each of the second plurality of visual stimuli has at least one of a second plurality of characteristics and is associated with a second plurality of coordinate locations and wherein a number of the second plurality of coordinate locations is less than a number of the first plurality of coordinate locations; causing each of the second plurality of visual stimuli to be displayed on said display in accordance with its one of the second plurality of coordinate locations and one of the second plurality of characteristics; receiving responses from the user, wherein the responses are indicative of the visual characteristics of the second plurality of visual stimuli experienced by the user; and determining attributes of the user's visual field based on the detected discrepancy and the responses that are indicative of the visual characteristics of the second plurality of visual stimuli experienced by the user.

Optionally, the discrepancy is indicative of one or more deficits in the visual field and is at least one of a partially missing vertical line, a partially missing horizontal line, a partially wavy vertical line, a partially wavy horizontal line, a partially blurred vertical line, or a partially blurred horizontal line.

Optionally, the method further comprises associating a first coordinate location from the first plurality of coordinate locations with the discrepancy and storing the detected discrepancy and first coordinate location as the first set of data. Optionally, the second plurality of coordinate locations are only positioned at the first coordinate location.

The grid may cover an entirety of the visual field of the user. The grid may be an Amsler grid. Optionally, grid is defined by at least five vertical lines intersecting at least five horizontal lines to create equally sized boxes.

Optionally, detecting the discrepancy is further achieved by a) receiving the response from the user, wherein the response is indicative of the visual characteristics of the first plurality of visual stimuli experienced by the user and b) comparing the visual characteristics of the first plurality of visual stimuli experienced by the user with the first plurality of characteristics to identify the discrepancy.

Optionally, at least one of the second plurality of coordinate locations, a temporal frequency of the second plurality of visual stimuli, or the second plurality of characteristics of the second plurality of visual stimuli presented to a left eye of the user differs from at least one of the second plurality of coordinate locations, a temporal frequency of the second plurality of visual stimuli, or the second plurality of characteristics of the second plurality of visual stimuli presented to a right eye of the user.

Optionally, the method further comprises using the first set of data to determine a first area in the visual field having a determined detection below a predefined value and a second area in the visual field having a determined detection above a predefined value. Optionally, the method further comprises setting a temporal frequency of the second plurality of visual stimuli in the first area different from a temporal frequency of the second plurality of visual stimuli in the second area.

Optionally, the method further comprises using the first set of data to determine a region that is smaller than the visual field of the user corresponding to an area of visual impairment. Optionally, the method further comprises presenting the second plurality of visual stimuli only within said region.

Optionally, the method further comprises using the determined attributes to identify one or more regions of visual impairment in the visual field. Optionally, the method further comprises generating a display wherein the display visually overlays the one or more regions onto the visual field.

The present specification also discloses a computer program product for evaluating a user's visual field and configured to be executed in a head-mounted device configured to be positioned on the user's head, wherein the device comprises at least one processor, a display in data communication with the at least one processor, and a non-transient memory in data communication with the at least one processor and adapted to store the computer program product, wherein, when executed, the computer program product is configured to evaluate the user's visual field by: generating a first plurality of visual stimuli, wherein the first plurality of visual stimuli is presented in a form of a grid defined by two or more vertical lines intersecting two or more horizontal lines, wherein the grid covers a first plurality of coordinate locations in the visual field, and wherein each of the first plurality of visual stimuli has at least one of a first plurality of characteristics; causing the first plurality of visual stimuli to be displayed on the display in accordance with its first plurality of characteristics; detecting a discrepancy based on a comparison of the first plurality of characteristics with a user's response that is indicative of the visual characteristics of the first plurality of visual stimuli experienced by the user; storing the detected discrepancy as a first set of data; using the first set of data to generate a second plurality of visual stimuli, wherein each of the second plurality of visual stimuli has at least one of a second plurality of characteristics and is associated with a second plurality of coordinate locations and wherein a number of the second plurality of coordinate locations is less than a number of the first plurality of coordinate locations; causing each of the second plurality of visual stimuli to be displayed on said display in accordance with its one of the second plurality of coordinate locations and one of the second plurality of characteristics; receiving responses from the user, wherein the responses are indicative of the visual characteristics of the second plurality of visual stimuli experienced by the user; and determining attributes of the user's visual field based on the detected discrepancy and the responses that are indicative of the visual characteristics of the second plurality of visual stimuli experienced by the user.

Optionally, the discrepancy is indicative of one or more deficits in the visual field and is at least one of a partially missing vertical line, a partially missing horizontal line, a partially wavy vertical line, a partially wavy horizontal line, a partially blurred vertical line, or a partially blurred horizontal line.

Optionally, the computer program product is further configured to associate a first coordinate location from the first plurality of coordinate locations with the discrepancy and store the detected discrepancy and first coordinate location as the first set of data. Optionally, the second plurality of coordinate locations are only positioned at the first coordinate location.

The grid may cover an entirety of the visual field of the user. The grid may be an Amsler grid. Optionally, the grid is defined by at least five vertical lines intersecting at least five horizontal lines to create equally sized boxes.

Optionally, detecting the discrepancy is further achieved by a) receiving the response from the user, wherein the response is indicative of the visual characteristics of the first plurality of visual stimuli experienced by the user and b) comparing the visual characteristics of the first plurality of visual stimuli experienced by the user with the first plurality of characteristics to identify the discrepancy.

Optionally, at least one of the second plurality of coordinate locations, a temporal frequency of the second plurality of visual stimuli, or the second plurality of characteristics of the second plurality of visual stimuli presented to a left eye of the user differs from at least one of the second plurality of coordinate locations, a temporal frequency of the second plurality of visual stimuli, or the second plurality of characteristics of the second plurality of visual stimuli presented to a right eye of the user.

Optionally, the computer program product is further configured to use the first set of data to determine a first area in the visual field having a determined detection below a predefined value and a second area in the visual field having a determined detection above a predefined value. Optionally, the computer program product is further configured to set a temporal frequency of the second plurality of visual stimuli in the first area different from a temporal frequency of the second plurality of visual stimuli in the second area.

Optionally, the computer program product is further configured to use the first set of data to determine a region that is smaller than the visual field of the user corresponding to an area of visual impairment. Optionally, the computer program product is further configured to present the second plurality of visual stimuli only within said region.

Optionally, the computer program product is further configured to use the determined attributes to identify one or more regions of visual impairment in the visual field. Optionally, The computer program product is further configured to generate a display wherein the display visually overlays the one or more regions onto the visual field.

The present specification also discloses a method of determining a value representative of a user's visual acuity using a head mounted device having a processor, memory, and a display, the method comprising: using the head mounted device, displaying a first plurality of visual stimuli having a first plurality of predefined visual characteristics; using the head mounted device, prompting the user to provide responses based on each of the first plurality of visual stimuli; using the head mounted device, receiving the responses indicative of one or more of the first plurality of predefined visual characteristics of each of the first plurality of visual stimuli; in the head mounted device and/or in a server in remote communication with the head mounted device, determining a first value indicative of a visual acuity of the user based on the responses; in the head mounted device and/or the server, determining if the first value is at or below a predefined threshold; if the first value is at or below the predefined threshold, displaying on the display or audibly communicating from the head mounted device an instruction to the user, wherein the instruction directs the user to increase a distance between the user and the display to create a new distance between the user and the display; determining the new distance; and using the head mounted device, displaying a second plurality of visual stimuli having a second plurality of predefined visual characteristics based at least in part on the new distance.

Optionally, the method further comprises using the head mounted device, prompting the user to provide new responses based on each of the second plurality of visual stimuli, receiving the new responses indicative of one or more of the second plurality of predefined visual characteristics of each of the second plurality of visual stimuli, and in the head mounted device and/or in the server, determining an updated value indicative of the visual acuity of the user based at least in part on the new responses. The updated value indicative of the visual acuity may be below the predefined threshold.

The predefined threshold may be in a range of 20/70 to 20/90. The predefined threshold may be 20/80.

Optionally, the predefined threshold is a function of the a resolution of the display.

Optionally, each of the first plurality of visual stimuli has similar visual characteristics of the first plurality of predefined visual characteristics, except are differently sized.

Optionally, each of the second plurality of visual stimuli has similar visual characteristics of the second plurality of predefined visual characteristics, except are differently sized.

Optionally, increasing the distance between the user and the display to create a new distance between the user and the display comprises removing the display from a head mounting apparatus and positioning it further from the user's eyes. The head mounted device may comprise a mobile phone positioned in a head mounting apparatus wherein the processor, the memory, and the display are components of the mobile phone.

The present specification also discloses a computer program product adapted to be stored and executed in a head-mounted device configured to be positioned on the user's head, wherein the device comprises at least one processor, a display in data communication with the at least one processor, a non-transient memory in data communication with the at least one processor and adapted to store the computer program product, and wherein the computer program product comprises a plurality of non-transient programmatic instructions that, when executed: cause a first plurality of visual stimuli having a first plurality of predefined visual characteristics to be displayed on the display; cause prompts to be presented to the user to provide responses based on each of the first plurality of visual stimuli; receive the responses indicative of one or more of the first plurality of predefined visual characteristics of each of the first plurality of visual stimuli; determine a first value indicative of a visual acuity of the user based on the responses; determine if the first value is at or below a predefined threshold; if the first value is at or below the predefined threshold, cause a display on the display or an audible communication, wherein the display or audible communication is an instruction to the user and wherein the instruction directs the user to increase a distance between the user and the display to create a new distance between the user and the display; determine the new distance; and cause a second plurality of visual stimuli having a second plurality of predefined visual characteristics to be displayed on the display based at least in part on the new distance.

Optionally, the plurality of non-transient programmatic instructions, when executed, further cause prompts to be presented to the user to provide new responses based on each of the second plurality of visual stimuli, receive the new responses indicative of one or more of the second plurality of predefined visual characteristics of each of the second plurality of visual stimuli, and determine an updated value indicative of the visual acuity of the user based at least in part on the new responses. The updated value indicative of the visual acuity may be below the predefined threshold.

The predefined threshold may be in a range of 20/70 to 20/90. The predefined threshold may be 20/80.

Optionally, the predefined threshold is a function of the a resolution of the display.

Optionally, each of the first plurality of visual stimuli has similar visual characteristics of the first plurality of predefined visual characteristics, except are differently sized.

Optionally, each of the second plurality of visual stimuli has similar visual characteristics of the second plurality of predefined visual characteristics, except are differently sized.

Optionally, increasing the distance between the user and the display to create a new distance between the user and the display comprises removing the display from a head mounting apparatus and positioning it further from the user's eyes.

The head mounted device may comprise a mobile phone positioned in a head mounting apparatus and wherein the processor, the memory, and the display are components of the mobile phone.

The present specification also discloses a method of evaluating a user's visual field using a head-mounted device configured to be positioned on the user's head, wherein the device comprises at least one processor, a display in data communication with the at least one processor, and a non-transient memory in data communication with the at least one processor and adapted to store programmatic instructions that, when executed, execute said method, the method comprising: generate a first plurality of visual stimuli, wherein each of the first plurality of visual stimuli is associated with one of a first plurality of coordinate locations in a visual field of the user and wherein each of the first plurality of visual stimuli has at least one of a first plurality of characteristics; causing each of the first plurality of visual stimuli to be displayed on said display in accordance with its one of a first plurality of coordinate locations and one of the first plurality of characteristics; determining if the user detects each of the first plurality of visual stimuli at each of the first plurality of coordinate locations; storing the determined detection as a first set of data; using said first set of data to generate a second plurality of visual stimuli, wherein each of the second plurality of visual stimuli is associated with one of a second plurality of coordinate locations, wherein each of the second plurality of visual stimuli has at least one of a second plurality of characteristics, and wherein a number of the second plurality of coordinate locations is less than a number of the first plurality of coordinate locations or a frequency of the first plurality of visual stimuli is different than a frequency of the second plurality of visual stimuli; and causing each of the second plurality of visual stimuli to be displayed on said display in accordance with its one of the second plurality of coordinate locations, its frequency and one of the second plurality of characteristics.

The present specification also discloses a method of determining a value representative of a user's visual acuity using a head mounted device having a processor, memory, and a display and/or a server remote from the head mounted device that is data communication with the head mounted device, the method comprising: using the head mounted device, displaying a plurality of visual trials, wherein each visual trial comprises a visual stimulus defined by a plurality of predefined visual characteristics, wherein, between each trial of the plurality of visual trials, each visual stimulus has the same plurality of predefined visual characteristics but are either differently sized or have different levels of contrast; using the head mounted device, prompting the user to provide a response indicative of characteristics of the visual stimulus in each of the plurality of visual trials; using the head mounted device, receiving the responses indicative of the characteristics of the visual stimulus in each of the plurality of visual trials; in the head mounted device and/or the server, applying a first fitting function to determine a first value indicative of a visual acuity of the user based on the first responses; and applying a second fitting function that is different from the first fitting function if the first value is equal to or above a visual acuity threshold value.

The number of trials in the plurality of trials may be in a range of 3 to 10.

Optionally, a level of contrast is at least one of the plurality of predefined visual characteristics of the plurality of visual stimuli that changes between each of the plurality of trials.

Optionally, each of the first responses defines an orientation or an identity of each of the plurality of visual stimuli.

Optionally, determining the first value indicative of the visual acuity of the user based on the first responses is achieved by fitting a function to the first responses and identifying the first value by mapping a threshold value to the fitted function.

Optionally, color or luminance is at least one of the plurality of predefined visual characteristics of the plurality of visual stimuli that changes between each of the plurality of trials.

Optionally, the plurality of visual stimuli comprises optotypes wherein the optotypes are at least one of: Landolt ‘C’, tumbling ‘E’ or Sloan letters.

Optionally, determining the final value indicative of the user's visual acuity comprises fitting a function to the first value and the plurality of additional values and mapping a threshold value to the fitted function. Optionally, the method further comprises replacing threshold parameters in a psychometric model with acuity relations from a fitted function to determine the user's visual acuity.

Optionally, the method further comprises determining the user's visual acuity by first displaying a Landolt ‘C’, tumbling ‘E’ or Snellen vision test using the head mounted device and, when the user's visual acuity is determined to be better than 20/80, executing each step of the method.

Optionally, the method further comprises calibrating the head mounted device based on one or more hardware properties of the head mounted display device, wherein the hardware properties are one or more of: non-square pixels, luminance resolution, display technology, colors of pixels, display gamma, edge sharpness, and luminance differences for gray levels.

The present specification also discloses a head-mounted device configured to determine a value representative of a user's visual acuity, the head-mounted device comprising: at least one processor; a display in data communication with the at least one processor; a non-transient memory in data communication with the at least one processor and adapted to store programmatic instructions that, when executed: display a plurality of visual stimuli having a plurality of predefined visual characteristics, wherein each of the plurality of visual stimuli has the same plurality of predefined visual characteristics but are differently sized; prompt the user to provide first responses indicative of characteristics of each of the plurality of visual stimuli; receive the first responses indicative of the characteristics of each of the plurality of visual stimuli; determine a first value indicative of a visual acuity of the user based on the first responses; perform a plurality of trials defined by the display, prompt, receive, and determine steps to determine a plurality of additional values, wherein each of the plurality of trials uses the same plurality of visual stimuli except at least one of the plurality of predefined visual characteristics of the plurality of visual stimuli changes between each of the plurality of trials; and determine a final value indicative of the user's visual acuity as a function of the first value and the plurality of additional values.

Optionally, a level of contrast is the at least one of the plurality of predefined visual characteristics of the plurality of visual stimuli that changes between each of the plurality of trials.

Optionally, each of the first responses defines an orientation or an identity of each of the plurality of visual stimuli.

Optionally, the executed programming instructions determine the first value indicative of the visual acuity of the user based on the first responses by fitting a function to the first responses and identifying the first value by mapping a threshold value to the fitted function.

Optionally, color or luminance is the at least one of the plurality of predefined visual characteristics of the plurality of visual stimuli that changes between each of the plurality of trials.

Optionally, the plurality of visual stimuli comprises optotypes wherein the optotypes are at least one of: Landolt ‘C’, tumbling ‘E’ or Sloan letters.

Optionally, the executed programmatic instructions determine the final value indicative of the user's visual acuity by fitting a function to the first value and the plurality of additional values and mapping a threshold value to the fitted function.

Optionally, the executed programmatic instructions further replace threshold parameters in a psychometric model with acuity relations from the fitted function to determine the user's visual acuity.

Optionally, the executed programmatic instructions further determine the user's visual acuity by first causing a Landolt ‘C’, tumbling ‘E’ or Sloan letter to be displayed and, when the programmatic instructions determine the user's visual acuity to be better than 20/80, the programmatic instructions execute the display, prompt, receive, determine, perform, and determine limitations.

Optionally, the executed programmatic instructions calibrate the head mounted display device based on one or more hardware properties of the head mounted display device, wherein the hardware properties are one or more of: non-square pixels, luminance resolution, display technology, colors of pixels, edge sharpness, and luminance differences for gray levels.

In some embodiments, the present specification describes a method of determining a value representative of a user's visual acuity using a head mounted device having a processor, memory, and a display and/or a server remote from the head mounted device that is data communication with the head mounted device, the method comprising: using the head mounted device, displaying a plurality of visual stimuli having a plurality of predefined visual characteristics, wherein each of the plurality of visual stimuli has the same plurality of predefined visual characteristics but are differently sized; using the head mounted device, prompting the user to provide first responses indicative of characteristics of each of the plurality of visual stimuli; using the head mounted device, receiving the first responses indicative of the characteristics of each of the plurality of visual stimuli; in the head mounted device and/or the server, determining a first value indicative of a visual acuity of the user based on the first responses; performing a plurality of trials defined by the generating, prompting, receiving, and determining steps to determine a plurality of additional values, wherein each of the plurality of trials uses the same plurality of visual stimuli except at least one of the plurality of predefined visual characteristics of the plurality of visual stimuli changes between each of the plurality of trials; and in the head mounted device and/or server, determining a final value indicative of the user's visual acuity as a function of the first value and the plurality of additional values.

Optionally, a contrast level is one of the plurality of predefined visual characteristics of the plurality of visual stimuli that changes between each of the plurality of trials.

Optionally, each of the responses defines an orientation or an identity of each of the plurality of visual stimuli.

Optionally, determining the first value indicative of the visual acuity of the user based on the first responses is achieved by fitting a function to the first responses and identifying the first value by mapping a threshold value to the fitted function.

Optionally, color or luminance is one of the plurality of predefined visual characteristics of the plurality of visual stimuli that changes between each of the plurality of trials.

Optionally, the plurality of visual stimuli comprises optotypes and the optotypes are at least one of: Landolt ‘C’, tumbling ‘E’ or Sloan letters.

Optionally, determining the final value indicative of the user's visual acuity comprises fitting a function to the first value and the plurality of additional values and mapping a threshold value to the fitted function.

Optionally, the method further comprises replacing threshold parameters in a psychometric model with acuity relations from the fitted function to determine the user's visual acuity.

Optionally, the method further comprises determining the user's visual acuity by first displaying a Landolt ‘C’, tumbling ‘E’ or Snellen vision test using the head mounted device and, when the user's visual acuity is determined to be better than 20/80, executing each step above.

Optionally, the method further comprises calibrating the head mounted device based on one or more hardware properties of the head mounted display device, wherein the hardware properties are one or more of: non-square pixels, luminance resolution, display technology, colors of pixels, edge sharpness, and luminance differences for gray levels.

In some embodiments, the present specification describes a head-mounted device configured to determine a value representative of a user's visual acuity, the head-mounted device comprising: at least one processor; a display in data communication with the at least one processor; a non-transient memory in data communication with the at least one processor and adapted to store programmatic instructions that, when executed: display a plurality of visual stimuli having a plurality of predefined visual characteristics, wherein each of the plurality of visual stimuli has the same plurality of predefined visual characteristics but are differently sized; prompt the user to provide first responses indicative of characteristics of each of the plurality of visual stimuli; receive the first responses indicative of the characteristics of each of the plurality of visual stimuli; determine a first value indicative of a visual acuity of the user based on the first responses; perform a plurality of trials defined by the display, prompt, receive, and determine steps to determine a plurality of additional values, wherein each of the plurality of trials uses the same plurality of visual stimuli except at least one of the plurality of predefined visual characteristics of the plurality of visual stimuli changes between each of the plurality of trials; and determine a final value indicative of the user's visual acuity as a function of the first value and the plurality of additional values.

Optionally, a level of contrast is the at least one of the plurality of predefined visual characteristics of the plurality of visual stimuli that changes between each of the plurality of trials.

Optionally, each of the first responses defines an orientation or an identity of each of the plurality of visual stimuli.

Optionally, the executed programming instructions determine the first value indicative of the visual acuity of the user based on the first responses by fitting a function to the first responses and identifying the first value by mapping a threshold value to the fitted function.

Optionally, color or luminance is the at least one of the plurality of predefined visual characteristics of the plurality of visual stimuli that changes between each of the plurality of trials.

Optionally, the plurality of visual stimuli comprises optotypes and the optotypes are at least one of: Landolt ‘C’, tumbling ‘E’ or Sloan letters.

Optionally, the executed programmatic instructions determine the final value indicative of the user's visual acuity by fitting a function to the first value and the plurality of additional values and mapping a threshold value to the fitted function.

Optionally, the executed programmatic instructions further replace threshold parameters in a psychometric model with acuity relations from the fitted function to determine the user's visual acuity.

Optionally, the executed programmatic instructions further determine the user's visual acuity by first causing a Landolt ‘C’, tumbling ‘E’ or Sloan letter to be displayed and, when the programmatic instructions determine the user's visual acuity to be better than 20/80, the programmatic instructions execute the display, prompt, receive, determine, perform, and determine limitations of the system.

Optionally, the executed programmatic instructions calibrate the head mounted display device based on one or more hardware properties of the head mounted display device, wherein the hardware properties are one or more of: non-square pixels, luminance resolution, display technology, colors of pixels, edge sharpness, and luminance differences for gray levels.

In some embodiments, the present specification discloses a method of evaluating a user's vision using a head-mounted device by combining results of one or more vision function tests (VFT) and Amsler grid tests taken by the user via a Head-mounted vision assist and diagnostic device (HMVD), the method comprising: making the user take the VFT and the Amsler grid test via the HMVD a predefined number of times and in a predefined order; obtaining results from each test taken by the user; combining the results to obtain a combined test result for the user; and diagnosing the user's vision condition based on the combined test result.

Optionally, the user is made to take the two tests alternately and repeatedly at different times. Optionally, a plurality of users are grouped based on one or more of: demographics, location, treating physician, eye condition or treatment protocol being followed, are made to take the VFT and the Amsler grid test via the HMVD.

Optionally, the results of the two tests are combined to obtain combined measurements comprising superimposed graphic overlay, showing interdependencies between timing of test, eye conditions and user groups.

Optionally, diagnosing the user's vision condition comprises obtaining information about a progression of the user's vision condition.

Optionally, the method further comprises diagnosing the vision conditions of the plurality of users by determining similarities and differences between vision conditions of different user groups based on the results of the tests conducted via the HMVD.

Optionally, the VFT and the Amsler grid tests are varied for improving diagnostics accuracy and localization of areas within the user's visual field with vision impairment, depending on a type of visual distortion.

Optionally, making the user take the VFT and the Amsler grid test via the HMVD comprises presenting one or more stimuli to the user via a display of the HMVD.

Optionally, eye tracking is used to present the stimuli to the user.

Optionally, the VFT and the Amsler grid tests are randomized by varying test parameters being one or more of: a degree of the user's visual angle; color, line thickness and resolution of the HMVD's display; ambient light; a presented stimulus; location of the presented stimulus; fixation targets; user's eye that is presented the stimulus.

Optionally, the combined test results are used to configure the HMVD with a filter that compensates for one or more of the user's identified visual conditions.

Optionally, the combined test results are used to configure the HMVADD for one or more of HMVD parameters.

Optionally, the parameters comprise screen brightness, resolution, distance from the eyes, lens, gamma, and ambient light.

In some embodiments, the present specification discloses a method of conducting an Amsler grid test on a user wearing Head-mounted vision assist and diagnostic device (HMVD), the method comprising: presenting to the user a grid like structure via the HMVD; prompting the user to mark a location and severity of vision impairment/distortion in the grid; prompting the user to provide a response indicative of a type of vision distortion experienced; analyzing the user's response by using one or more analytical tools to obtain the user's vision test results; and communicating the test results to the user.

Optionally, the presented grid like structure comprises lines, checkerboard patterns or common objects with straight lines known by the user.

Optionally, the user is prompted to mark a location of vision impairment by marking a grid coordinate using one or more of: voice commands, a joystick coupled with the HMVD, or gestures recognized by the HMVD via gyro sensors coupled with the HMVD.

Optionally, the user is prompted to mark a severity of vision impairment by using a number rating on a predefined scale presented via the HMVD.

Optionally, marking a severity of vision impairment further comprises identifying a type of vision distortion by using predefined keywords or by selecting a suitable response from a plurality of options presented via the HMVD.

In some embodiments, the present specification discloses a method of conducting a visual field test (VFT) on users wearing Head-mounted vision assist and diagnostic device (HMVD), by using stored data regarding the users and one or more previous vision tests undertaken by the users, said data being recorded and stored via the HMVADD, the method comprising testing the users' visions for existing scotomas and testing the users' vision for formation of new scotomas. Optionally, the method further comprises obtaining results of the VFT conducted and using the test results to configure one or more HMVADD with a gravity lens filter.

The present specification also discloses a method of evaluating a user's peripheral vision using a head-mounted device configured to be positioned on the user's head, wherein the device comprises at least one processor, a display in data communication with the at least one processor, a non-transient memory in data communication with the at least one processor and adapted to store programmatic instructions that, when executed, execute said method, the method comprising: generate a first plurality of visual stimuli, wherein each of the first plurality of visual stimuli is associated with one of a first plurality of coordinate locations in a visual field of the user and wherein each of the first plurality of visual stimuli has at least one of a first plurality of characteristics; causing each of the first plurality of visual stimuli to be displayed on said display in accordance with its one of a first plurality of coordinate locations and one of the first plurality of characteristics; determining if the user detects each of the first plurality of visual stimuli at each of the first plurality of coordinate locations; storing the determined detection as a first set of data; using said first set of data to generate a second plurality of visual stimuli, wherein each of the second plurality of visual stimuli is associated with one of a second plurality of coordinate locations, wherein each of the second plurality of visual stimuli has at least one of a second plurality of characteristics, and wherein a number of the second plurality of coordinate locations is less than a number of the first plurality of coordinate locations or a frequency of the first plurality of visual stimuli is different than a frequency of the second plurality of visual stimuli; and causing each of the second plurality of visual stimuli to be displayed on said display in accordance with its one of the second plurality of coordinate locations, its frequency and one of the second plurality of characteristics.

Optionally, the second plurality of coordinate locations are only positioned at the first plurality of coordinate locations where the determined detection of the first plurality of visual stimuli is indicative of one or more deficits in the visual field of the user.

Optionally, at least one of the second plurality of coordinate locations, the frequency of the second plurality of visual stimuli, or the second plurality of characteristics of the second plurality of visual stimuli presented to a left eye of the user differs from at least one of the second plurality of coordinate locations, the frequency of the second plurality of visual stimuli, or the second plurality of characteristics of the second plurality of visual stimuli presented to a right eye of the user.

Optionally, the determined detection is determined by receiving user responses and determining if the user responses correspond with the first plurality of visual to a predetermined value.

Optionally, the method further comprises using the first set of data to determine a first area in the visual field of the user corresponding to one or more boundaries between areas of determined detection below the predefined value and areas of determined detection above the predefined value. Optionally, the method further comprises increasing the frequency of the second plurality of visual stimuli in the first area relative to the frequency of the first plurality of visual stimuli.

Optionally, the first plurality of visual stimuli is generated in a first session adapted to evaluate the user's peripheral vision and the second plurality of visual stimuli is generated in a second session adapted to evaluate the user's peripheral vision, and wherein the first and second sessions occur at different points in time.

Optionally, the method further comprises using the first set of data to determine a region internal to an area in the visual field of the user corresponding to an area of determined detection below a predefined value. Optionally, the method further comprises decreasing the frequency of the second plurality of visual stimuli in the region relative to the frequency of the first plurality of visual stimuli.

Optionally, the method further comprises using the first set of data to determine a region internal to an area in the visual field of the user corresponding to an area of determined detection above a predefined value. Optionally, the method further comprises decreasing the frequency of the second plurality of visual stimuli in the region relative to the frequency of the first plurality of visual stimuli.

Optionally, the method further comprises using the first set of data to generate the second plurality of visual stimuli, wherein the second plurality of visual stimuli have a different temporal profile than the first plurality of visual stimuli.

Optionally, the method further comprises generating a visual grid having one or more images in one or more cells of the visual grid; prompting the user to identify one or more anomalies in the one or more images; and generating a second set of data representative of in which of the one or more cells the user identified one or more anomalies. Optionally, the method further comprises causing a visual representation of the first set of data and a visual representation of the second set of data to be concurrently displayed. Optionally, the visual representation of the first set of data is overlaid on the visual representation of the second set of data or the visual representation of the second set of data is overlaid on the visual representation of the first set of data.

Optionally, the method further comprises using the first set of data together with the second set of data to generate the second plurality of visual stimuli. Optionally, each of the second plurality of coordinate locations associated with each of the second plurality of visual stimuli is determined based on a coordinate of the one or more anomalies in the visual grid. Optionally, the second plurality of visual stimuli are only positioned in locations corresponding to coordinates of the one or more anomalies in the visual grid.

Optionally, the first set of data is representative of areas in the visual field having the determined detection below a predefined value and the second set of data is representative of areas in the visual field having visual anomalies.

The present specification also discloses a computer program product adapted to be stored and executed in a head-mounted device configured to be positioned on the user's head, wherein the device comprises at least one processor, a display in data communication with the at least one processor, a non-transient memory in data communication with the at least one processor and adapted to store the computer program product, and wherein the computer program product comprises a plurality of non-transient programmatic instructions that, when executed: generate a first plurality of visual stimuli, wherein each of the first plurality of visual stimuli is associated with one of a first plurality of coordinate locations in a visual field of the user and wherein each of the first plurality of visual stimuli has at least one of a first plurality of visual characteristics; cause each of the first plurality of visual stimuli to be displayed on said display in accordance with its one of a first plurality of coordinate locations and one of the first plurality of visual characteristics; determine a detection accuracy at each of the first plurality of coordinate locations; store the determined detection accuracy as a first set of data; use the first set of data to generate a second plurality of visual stimuli, wherein each of the second plurality of visual stimuli is associated with one of a second plurality of coordinate locations, wherein each of the second plurality of visual stimuli has at least one of a second plurality of visual characteristics, and wherein a number of the second plurality of coordinate locations is less than a number of the first plurality of coordinate locations or a frequency of the first plurality of visual stimuli is different than a frequency of the second plurality of visual stimuli; and cause each of the second plurality of visual stimuli to be displayed on said display in accordance with its one of the second plurality of coordinate locations, its frequency and one of the second plurality of visual characteristics.

Optionally, the second plurality of coordinate locations are only positioned at the first plurality of coordinate locations where the determined detection accuracy of the first plurality of visual stimuli is indicative of one or more deficits in the visual field of the user.

Optionally, at least one of the second plurality of coordinate locations, the frequency of the second plurality of visual stimuli, or the second plurality of visual characteristics of the second plurality of visual stimuli presented to a left eye of the user differs from at least one of the second plurality of coordinate locations, the frequency of the second plurality of visual stimuli, or the second plurality of visual characteristics of the second plurality of visual stimuli presented to a right eye of the user.

Optionally, when executed, the plurality of non-transient programmatic instructions further determine the detection accuracy by receiving user responses and determining if the user responses correspond with the first plurality of visual to a predetermined value.

Optionally, when executed, the plurality of non-transient programmatic instructions further use the first set of data to determine a first area in the visual field of the user corresponding to one or more boundaries between areas of detection accuracy below the predefined value and areas of detection accuracy above the predefined value. Optionally, when executed, the plurality of non-transient programmatic instructions further increase the frequency of the second plurality of visual stimuli in the first area relative to the frequency of the first plurality of visual stimuli.

Optionally, when executed, the plurality of non-transient programmatic instructions further use the first set of data to determine a region internal to an area in the visual field of the user corresponding to an area of detection accuracy below a predefined value. Optionally, when executed, the plurality of non-transient programmatic instructions further decrease the frequency of the second plurality of visual stimuli in the region relative to the frequency of the first plurality of visual stimuli.

Optionally, when executed, the plurality of non-transient programmatic instructions further use the first set of data to determine a region internal to an area in the visual field of the user corresponding to an area of detection accuracy above a predefined value. Optionally, when executed, the plurality of non-transient programmatic instructions further decrease the frequency of the second plurality of visual stimuli in the region relative to the frequency of the first plurality of visual stimuli.

Optionally, when executed, the plurality of non-transient programmatic instructions further use the first set of data to generate the second plurality of visual stimuli, wherein the second plurality of visual stimuli have a different temporal profile than the first plurality of visual stimuli.

Optionally, when executed, the plurality of non-transient programmatic instructions further: generate a visual grid having one or more images in one or more cells of the visual grid;

prompt the user to identify one or more anomalies in the one or more images; and generate a second set of data representative of in which of the one or more cells the user identified one or more anomalies. Optionally, when executed, the plurality of non-transient programmatic instructions further cause a visual representation of the first set of data and a visual representation of the second set of data to be concurrently displayed.

Optionally, when executed, the plurality of non-transient programmatic instructions further cause the visual representation of the first set of data to be overlaid on the visual representation of the second set of data or the visual representation of the second set of data to be overlaid on the visual representation of the first set of data.

Optionally, when executed, the plurality of non-transient programmatic instructions further use the first set of data together with the second set of data to generate the second plurality of visual stimuli. Optionally, each of the second plurality of coordinate locations associated with each of the second plurality of visual stimuli is determined based on a coordinate of the one or more anomalies in the visual grid. Optionally, the second plurality of visual stimuli are only positioned in locations corresponding to coordinates of the one or more anomalies in the visual grid.

Optionally, the first set of data is representative of areas in the visual field having the detection accuracy below a predefined value and the second set of data is representative of areas in the visual field having visual anomalies.

The present specification also discloses a head-mounted device configured to determine a value representative of a user's visual acuity, the head-mounted device comprising: at least one processor; a display in data communication with the at least one processor; a non-transient memory in data communication with the at least one processor and adapted to store programmatic instructions that, when executed: display a plurality of visual stimuli having a plurality of predefined visual characteristics, wherein each of the plurality of visual stimuli has the same plurality of predefined visual characteristics but are differently sized; prompt the user to provide first responses indicative of characteristics of each of the plurality of visual stimuli; receive the first responses indicative of the characteristics of each of the plurality of visual stimuli; determine a first value indicative of a visual acuity of the user based on the first responses; perform a plurality of trials defined by the display, prompt, receive, and determine steps to determine a plurality of additional values, wherein each of the plurality of trials uses the same plurality of visual stimuli except at least one of the plurality of predefined visual characteristics of the plurality of visual stimuli changes between each of the plurality of trials; and determine a final value indicative of the user's visual acuity as a function of the first value and the plurality of additional values.

The aforementioned and other embodiments of the present specification shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings:

FIG. 1A illustrates an exemplary embodiment of a user-controllable vision-testing system on a user, in accordance with some embodiments of the present specification;

FIG. 1B illustrates a smartphone that may be used in the system of FIG. 1A, in accordance with some embodiments of the present specification;

FIG. 1C illustrates body of a goggle that may be used in the system of FIG. 1A, in accordance with some embodiments of the present specification;

FIG. 1D illustrates an exemplary system environment including devices that may communicate with the system of FIG. 1A, in accordance with some embodiments of the present specification;

FIG. 1E illustrates a block drawing of the components of an exemplary system of FIG. 1A, in accordance with some embodiments of the present specification;

FIG. 1F illustrates a functional diagram illustrating a relationship of the functional modules of the integrated vision-assist software platform, in accordance with some embodiments of the present specification;

FIG. 1G illustrates a block drawing of the components of an exemplary vision diagnostic system, in accordance with some embodiments of the present specification;

FIG. 2A is a flow chart illustrating a method of extrapolating a user's vision acuity level to a desired level by using a head mounted display system, in accordance with some embodiments of the present specification;

FIG. 2B is a graph illustrating the relationship between stimulus size and contrast, in accordance with an embodiment of the present specification;

FIG. 3A is a flow chart illustrating a process of extrapolating visual acuity by using a head mounted display system, in accordance with some other embodiments of the present specification;

FIG. 3B is a flow chart illustrating another method of determining a user's vision acuity in a head mounted display system, in accordance with some embodiments of the present specification;

FIG. 4 is a block diagram of a remote management system which may be used to control the head-mounted vision device, in accordance with an embodiment of the present specification;

FIG. 5 presents a series of exemplary two pixel optotypes;

FIG. 6 presents a series of exemplary three pixel optotypes;

FIG. 7 presents a process for calibrating a new optotype relative to a known optotype;

FIG. 8 is a flowchart illustrating exemplary steps of executing an asynchronous diagnosis feature in the vision assist device, in accordance with an embodiment of the present specification;

FIG. 9 is a flowchart illustrating the steps of conducting an Amsler grid test by using an HMVADD, in accordance with an embodiment of the present specification;

FIG. 10 is a flowchart illustrating the steps of conducting a combination of Amsler grid test and VFT by using an HMVD, in accordance with an embodiment of the present specification; and

FIG. 11 illustrates a VFT test data being combined or overlaid with Amsler grid test data, in accordance with some embodiments of the present specification.

### DETAILED DESCRIPTION

Embodiments of the present specification provide systems and methods for providing vision assistance as well as evaluating vision acuity of a user. In various embodiments, the device of the present specification may be used for one or more of: providing vision assistance to users and/or diagnosing vision related problems.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

In the description and claims of the application, the term “user” is broadly used to describe an individual who is using, and is the subject of, the vision evaluation methods and systems described herein. The term “user” shall encompass terms such as patient, individual, or vision-impaired individual.

In the description and claims of the application, the term “trial” refers to a single instance of presenting one visual stimulus to a user and receiving a response from the user specific to that one visual stimulus. A vision test typically comprises more than one trial, preferably in a range of 3 to 7 trials.

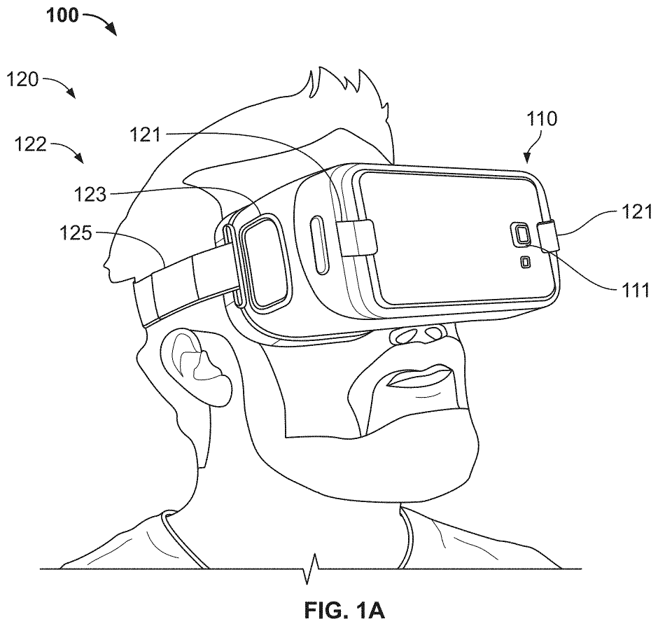

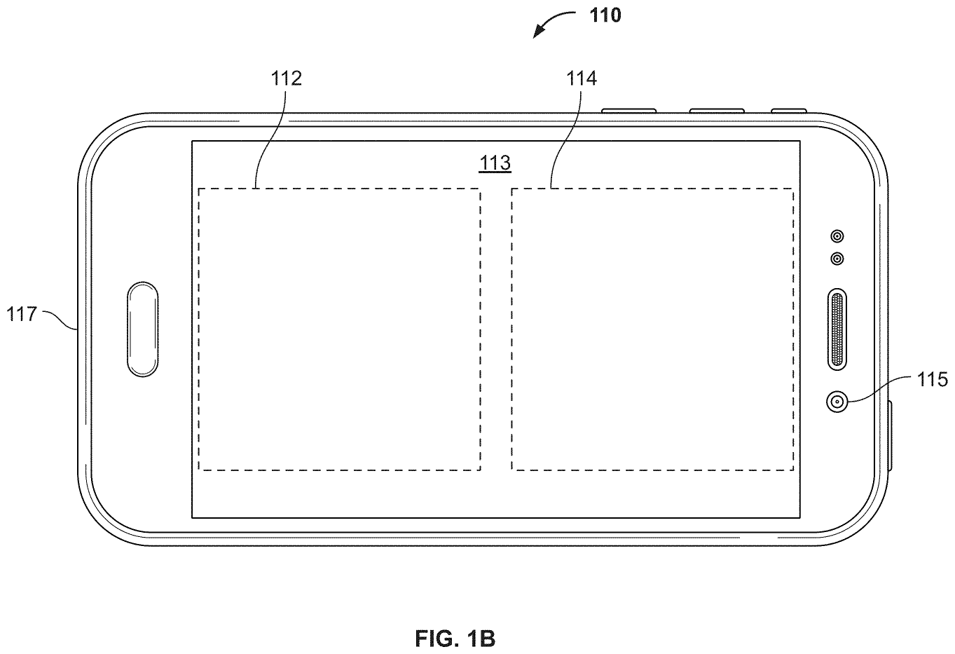

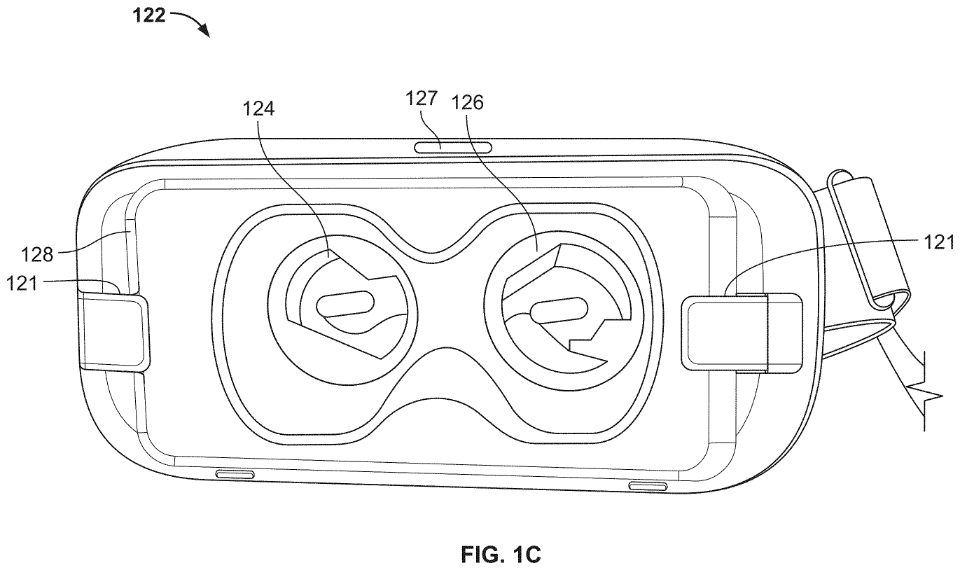

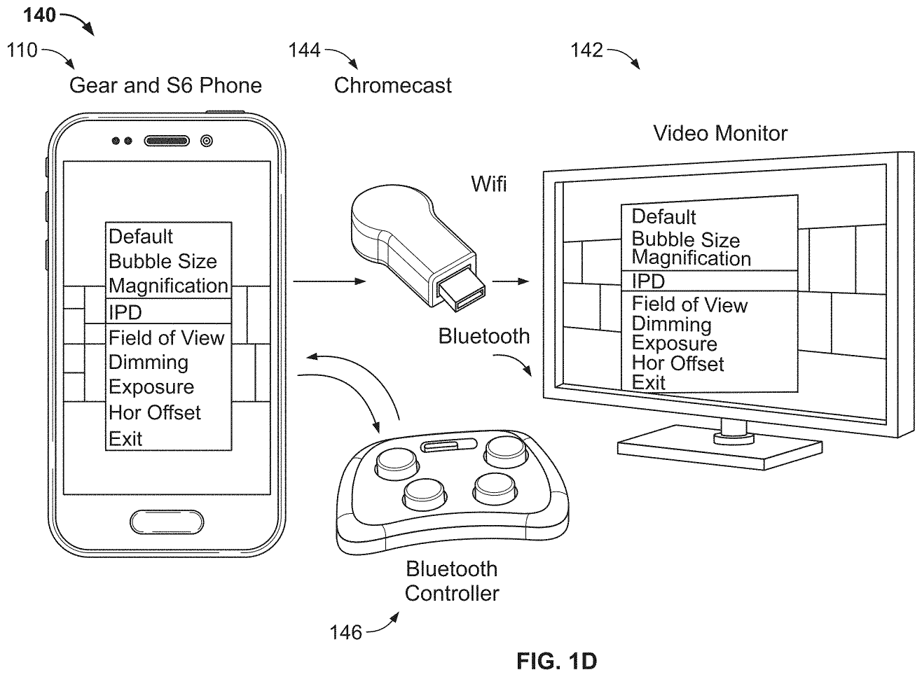

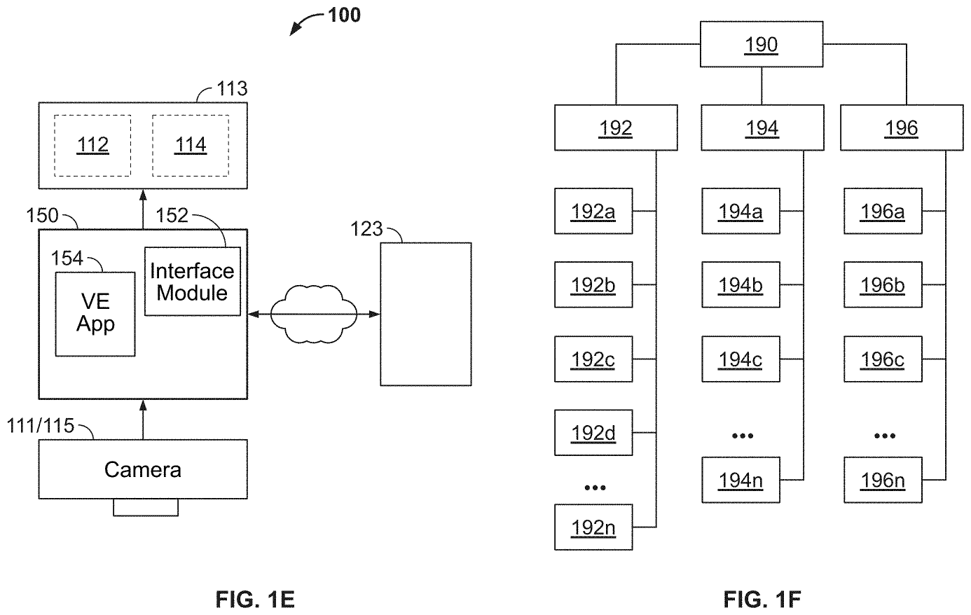

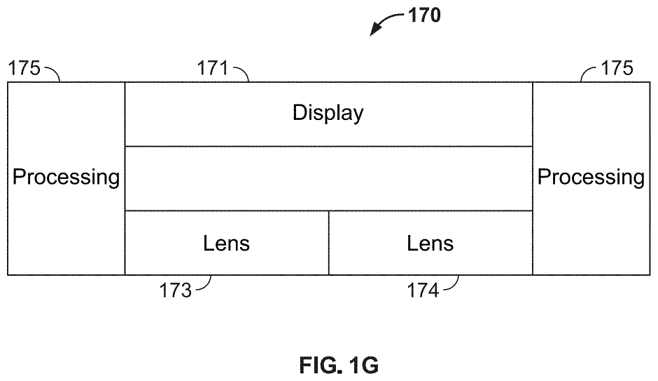

By way of a specific embodiment, FIGS. 1A, 1B, and 1C show a first embodiment user-controllable vision assist and/or diagnostic system 100, where FIG. 1A shows the system on a user, FIG. 1B shows a smartphone used in the system and FIG. 1C shows the body of the goggles used in the system. It should be appreciated that the embodiments described herein encompass a head-mounted device that may be used to both diagnose, and then to treat, a user or augment the vision of a user and/or a head-mounted device that may be used to just diagnose one or more conditions of the user, including visual acuity. The embodiments described herein may be universally referred to as head-mounted vision devices (HMVD).

System 100 includes a smartphone 110 and pair of goggles 120. Smartphone 110 includes the electronics necessary for the vision-assist system 100, including a processor and memory, an optional forward facing camera 111, as shown in FIG. 1A, and a screen 113 on the side opposite of the camera, as shown in FIG. 1B. Smartphone 110 also includes an electrical connector 117 and may also include a backward facing camera 115, which may be used in certain embodiments. As described subsequently, computer generated images, such as optotypes, or processed camera images may be displayed on one portion of screen 113 shown as a left area 112 and a second portion of the screen is shown as right area 114. Smartphone 110 may further comprise a plurality of programmatic instructions which, when executed, implement one or more of the functional modules described herein and shown in, among other places, FIG. 1F.

Goggles 120 include a body 122 and a strap 125 for holding the goggles on the user's head and a connector 128 that mates with smartphone connector 117. Body 122 includes, as shown in FIG. 1A, a pair of clamps 121 for removably restraining smartphone 110 and making the electrical connection between connectors 117 and 128, and input device 123 for providing input to the smartphone through the connectors and, as shown in FIG. 1C, a left lens 124 and right lens 126 and, optionally, a focusing wheel 127. It should be appreciated that, when the device is a purely diagnostic system, it may comprise a display but no focusing wheel or additional lenses since a user would be expected to wear the corrective lenses he or she would normally wear. Alternatively, the device may further comprise a space, gap, or other receiving section that may be used to accept one or more lenses, or focusing elements, that have the focal properties of lenses the user would be prescribed by a clinician. In one embodiment, any lenses deployed in the HMVD, or determined to be optimal for a user of the HMVD, would be characterized by a focal length that is less than a focal length of lenses the user would otherwise use outside the HMVD, since the maximum focal length in the HMVD is limited by display positioning and the overall dimensions of the HMVD. Accordingly, the HMVD system may be configured to generate a prescription or determination of a preferred lens type for a user by automatically adjusting or correcting for the truncated maximum focal length.

When assembled as in FIG. 1A, with smartphone 110 held in place by clamps 121, system 100 presents what is displayed in area 112 of screen 113, through lens 124, to the user's left eye, and what is displayed in area 114 of the screen, through lens 126, to the user's right eye. The user may use the optional focusing wheel 127 to adjust the focus. In some embodiments, there may be one or more knobs, wheels, buttons, or other mechanisms to adjust the relative distance between right and left lenses and, therefore account for variations in interpupillary distance between users. There may additionally be one or more knobs, wheels, buttons, or other mechanisms to adjust the relative distance between a user's eyes and the display itself. In certain embodiments, goggles 120 are adapted to accept user input from input device 123, which may control or otherwise provide inputs to the accepted smartphone 110.

In embodiments, smartphone 110 is provided with programming, as through a vision-assist application (referred to herein as a “VE App”) which can: operate camera 111 in a video mode to capture a stream of “input images”; perform image processing on each input image to generate a stream of “output images”; and present the stream of output images to screen 113. In certain embodiments, each of the stream of output images is presented sequentially side-by-side as two identical images—one in area 112 and one in area 114. Further, it is preferred that vision-assist system 100 operates so that the time delay between when the input images are obtained and when the output images are provided to screen 113, is as short as possible so that a user may safely walk and interact with the environment with goggles 120 covering their eyes.

In certain embodiments, the VE App may also provide a menu of options that allow for the modification of how vision-assist system 100 processes and generates an output image from an input image. Thus, for example, vision-assist system 100 may execute image-processing algorithms having parameters, where the parameters are changeable through the menu by, for example, setting parameter values for magnification, or the size and shape of magnification of the output image.

Vision-assist system 100 has adjustable features that allow it to match the physiology of the user for use in different settings. These features are generally set once for each user, possibly with the need for periodic adjustment. Thus, for example, given the spacing between screen 113 and the eyes of user U, focusing wheel 127 permits for an optimal setting of the distance to lens 124 and 126. In addition, lens 124 and/or 126 may include refractive error correction. Further, it is important that the viewed spacing between the images in areas 112 and 114 match the user's Inter Pupillary Distance (IPD). This may be accounted for, by example, by shifting the spacing of the output images in areas 112 and 114 to match the IPD.

In various embodiments, the user may adjust setting using input device 123, which may be a touchpad, and which is electrically connected to smartphone 110, which is further programmed to modify the VE App according to such inputs; a Bluetooth game controller that communicates with the smartphone 110 via Bluetooth; voice control using the microphone of the phone; or gesture control using available devices such as the NOD gesture control ring.

In addition, there are other features of vision-assist system 100 that can either be set up once for a user or may be user-adjustable. These features may include, but are not limited to, adjustments to the magnitude, shape, size, or placement of minified or magnified portions of the output image, and color enhancement functions such as contrast, blur, ambient light level or edge enhancement of the entire image or portions of the image. In some embodiments, color and color sensitivity may be set or adjusted based on a plurality of different controls. In some embodiments, color and/or color sensitivity may be adjusted by the user. In some embodiments, color and/or color sensitivity adjustments are performed by the system to optimize colors. In other embodiments, the compass and/or accelerometers within smartphone 110 may be used for enhancing orientation, location, or positioning of output images.

In certain embodiments, sound and/or vibration may be provided on smartphone 110 to generate for proximity and hazard cues. In other embodiments, the microphone of smartphone 110 can be used to enter voice commands to modify the VE App. In certain other embodiments, image stabilization features or programming of smartphone 110 are used to generate output images.

In one embodiment, by way of example only, goggles 120 are commercially available virtual-reality goggles, such as Samsung Gear VR (Samsung Electronics Co. Ltd., Ridgefield Park, N.J.) and smartphone 110 is a Galaxy Note 4 (Samsung Electronics Co. Ltd., Ridgefield Park, N.J.). The Samsung Gear VR includes a micro USB to provide an electrical connection to the Galaxy Note 4 and has, as input devices 123, a touch pad and buttons.

It will be understood by those in the field that vision-assist system 100 may, instead of including a combination of smartphone and goggles, be formed from a single device which includes one or more cameras, a processor, display device, and lenses that provide an image to each eye of the user. In an alternative embodiment, some of the components are head-mounted and the other components are in communication with the head-mounted components using wired or wireless communication. Thus, for example, the screen and, optionally the camera, may be head-mounted, while the processor communicates with the screen and camera using wired or wireless communication. In such an embodiment, an integrated processor and memory would comprise a plurality of programmatic instructions which, when executed, implement one or more of the functional modules described herein and shown in, among other places, FIG. 1F.

Further, it will be understood that other combinations of elements may form the vision-assist system 100. Thus, an electronic device which is not a smartphone, but which has a processor, memory, camera, and display may be mounted in goggles 120. Alternatively, some of the electronic features described as being included in smartphone 110 may be included in goggles 120, such as the display or communications capabilities. Further, the input control provided by input device 123 may be provided by a remote-control unit that is in communication with smartphone 110.

FIG. 1E illustrates an exemplary set of system components that may be incorporated in the vision assist system 100, in accordance with some embodiments of the present specification. In embodiments, the system components are integrated with a pair of smart goggles, or a smartphone with an attachment that enables a user to wear the smartphone like goggles. In embodiments, the system components of vision assist system 100 may communicate with a user interface 123 that is integrated with its components, or externally connected to the system 100 through a wired or a wireless communication means. In some embodiments, the interface is one or a combination of a touchpad, a voice interface, an optical interface, a motion or gesture sensor, or any other type of interface. Camera 111 and backward camera 115 are configured to receive images, which are processed by a processing unit 150. In some embodiments, processing unit 150 comprises one or more software modules, including an interface module 152 and VE App 154, which include a programmed set of instructions for processing the input signals received by the interface module 152 from the user interface, in accordance with the instructions of the VE App 154.

In some embodiments, execution of a plurality of sequences of programmatic instructions or code enables or causes the CPU of the processing unit 150 to perform various functions and processes. The processing unit 150 may be any computing device having one or more processors and one or more computer-readable storage media such as RAM, hard disk or any other optical or magnetic media. In alternate embodiments, hard-wired circuitry may be used in place of, or in combination with, software instructions for implementation of the processes of systems and methods described in this application. Thus, the systems and methods described are not limited to any specific combination of hardware and software.

The term ‘module’ used in this disclosure may refer to computer logic utilized to provide a desired functionality, service or operation by programming or controlling a general purpose processor. In various embodiments, a module can be implemented in hardware, firmware, software or any combination thereof. The module may be interchangeably used with unit, logic, logical block, component, or circuit, for example. The module may be the minimum unit, or part thereof, which performs one or more particular functions.

FIG. 1D illustrates, without limitation, one embodiment of a clinical setup 140 that a clinician may use to configure vision-assist system 100. Clinical setup 140 may allow a user or a clinician to determine and setup the VE App by setting an IPD, the field of view (FoV), background dimming, ambient light level, as well as parameters that are also user-adjustable, such as the size, shape, magnification, and location of enhanced vision features, such as the magnification bubble described subsequently. The setup may also be used by the user and/or the clinician, or any other medical or non-medical person caring for the user, to diagnose the user's vision and configure the VE App to assist the user with and/or provide therapeutic treatments to the user for, user-specific vision-related issues.

Clinical setup 140 thus allows for the adjustment of parameters within, or used by, the VE App that smartphone 110 runs to implement the vision-assist system 100. Clinical setup 140 includes a monitor 142, a Wi-Fi device 144 to allow screen 113 of smartphone 110 to be displayed on the monitor, and a Bluetooth controller 146 to communicate via Bluetooth with smartphone 110. In general, clinical setup 140 accepts a video output from smartphone 110 of display 113, and projects what the user would see when using vision-assist system 110 on monitor 142.

In certain embodiments, features or aspects of the present vision-assist system 100 may be adjusted by a clinician using clinical setup 140. Using the vision-assist system 100, screen 113 of smartphone 110 is mirrored on monitor 142, using Wi-Fi device 144, for example, so that the clinician can view what the user is viewing in vision-assist system 100. In embodiments, Wi-Fi device 144 may refer to any other type of communication device enabling communication between the vision enhancement system 100 and one or more remote computing devices. The VE App on smartphone 110 includes a menu that allows for the selection of certain parameters that operate vision-assist system 100.

The clinician has access to the commands in the menu of the VE App via remote Bluetooth controller 146. In this way, the clinician can “tune” the device to the specific visual demands of the user.

In certain embodiments, Wi-Fi device 144 can be used to remotely add, augment or modify functions that allow vision-assists, mirror the display, monitor and control VE App configurations in a clinical environment. In certain embodiments, Bluetooth controller 146 can be used to control or modify visual enhancement functions. In certain other embodiments, the VE App may be reconfigured in a purely magnified format, making it possible for the low vision user to place phone calls, utilize maps, read announcements and perform all visual functions currently available to those with normal vision.

In embodiments, vision enhancement system 100 enables a user to choose a specialized processing for its operation, each specialized processing offering vision solutions for different types of user requirements. Referring to FIG. 1F, through a first graphical user interface (GUI) in a menu 190, a user may be able to select one of several modes of operation, 192, 194, or 196. In a first mode, an assistive mode of operation 192 is provided. In a second mode, a diagnostic mode of operation 194 is provided. In a third mode, a therapeutic mode of operation 196 is provided. A user may switch between different modes by using a button provided through an interface on screen 113 of smartphone 110, on monitor 142, or through any other type of interface in communication with the vision enhancement system 100. The assistive mode of operation 192 may have a number of different functions 192a-192n which actively improve, tailor, or customize a display to correct for the vision deficiencies of a user by, for example, modulating brightness, contrast, size, field of view, or any other of a plurality of display characteristics. The therapeutic mode of operation 106 may have a number of different functions 196a-196n which present one or more visual activities to help an individual's vision improve. Finally, the diagnostic mode of operation 1944 may have a number of different functions 194a-194n which present one or more visual tests to help determine the visual acuity of an individual and/or profile the vision characteristics of the individual, including field of view, peripheral vs. central visual accuracy, color blindness, ocular motility, depth perception, eye alignment, light reflection, and/or refraction testing.

In another embodiment, the HMVD 170 is a diagnostic system comprising a display 171, lenses 173, 174, and a processing system 175 configured to generate one or more images of the optotypes described herein. It should be appreciated that the optotype generation and processing system, used to assess visual acuity, is implemented in one or more software modules, comprising a plurality of programmatic instructions, stored in a memory of the processing system 175 and executed by one or more processors in the processing system 175.

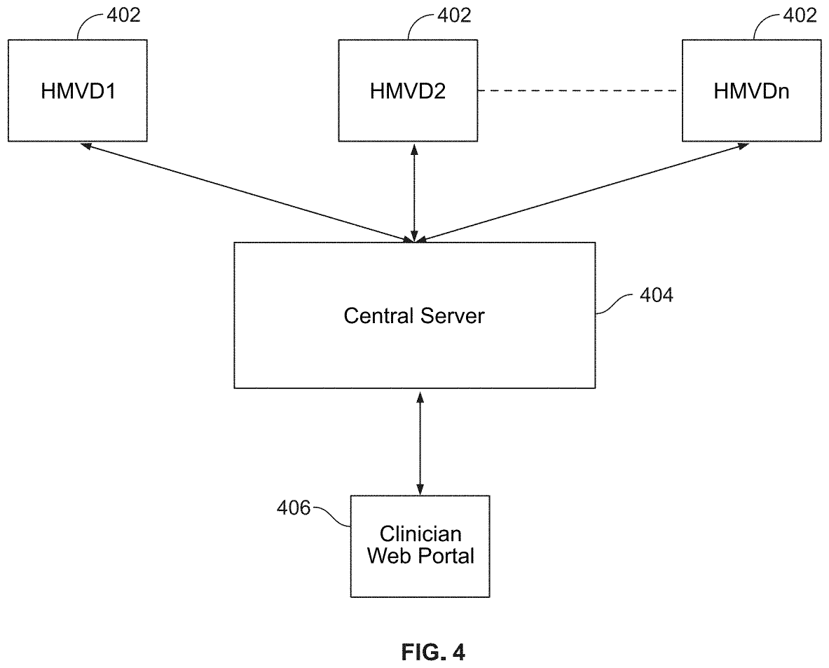

It should further be appreciated that the HMVD may be controlled remotely using a remote management system. FIG. 4 is a block diagram of the HMVD remote management system, in accordance with an embodiment of the present specification. In embodiments, a plurality of HMVDs 402 located at multiple user locations are communicatively coupled with a central server 404 located remotely from said user locations. In an embodiment, the central server 404 enables data to be bi-directionality sent to, and received from, each HMVD 402 and further enables such data to be accessed by one or more clinicians via a web portal 406 for monitoring said data for detecting vision problems, diagnosing vision-related conditions, and/or directing a user through vision therapies and/or diagnostic steps. The central server 204 may further comprise a plurality of programmatic instructions which, when executed, implement one or more of the functional modules described herein and shown in, among other places,

In an embodiment the present specification provides methods and systems for generating one or more visual acuity tests. Referring to FIG. 1G, the HMVD 170 may be configured to display a plurality of optotypes on the display 171 in a virtual-reality type setting such that it appears to be a fixed distance away from the viewer and wherein the apparent fixed distance is greater than the actual distance between the display 171 and the viewer's eyes. In such an embodiment, a plurality of optotypes may be generated by the processing system 175 and then transmitted to the display 171. Such optotypes may include those optotypes typically found in a Snellen chart or a Pelli-Robson chart, or any optotype in which more than one image is displayed concurrently and in which crowding effects between neighboring optotype images may affect the user's visual assessment of the images. Additionally, the HMVD 170 processing system 175 comprises one or more input mechanisms, such as buttons, knobs, switches, or connectivity to a second computing device through which input data may be provided and communicated to the processing system 175. These input mechanisms are configured to receive data indicative of a desired degree of apparent distance between the viewer and the displayed optotypes, a desired degree of luminance, a desired degree of brightness, and/or a desired level of contrast and then modify the display 171 according to the desired data.

Additionally, or alternatively, the HMVD may be configured to assess the visual acuity of a user in a non-virtual reality type setting, that is where the apparent distance and actual distance of the display are substantially equivalent. In such embodiments, the diagnostic capabilities of a HMVD may be limited by the display resolution, as previously explained, and additional techniques have to be implemented to assess a user who may have a visual acuity that is better than 20/80. In one embodiment, an improved approach may be effectuated by implementing a first visual acuity test using optotypes evaluated in using a one-dimensional scale until the system determines the individual wearing the head mounted display has a visual acuity better than a threshold value, such as 20/80, at which point the system switches to using a second visual acuity test that uses optotypes evaluated in a greater than one-dimensional scale, such as a two-dimensional scale or more dimensions. Additionally, or alternatively, the second visual acuity test may be used from the outset, without performing or switching from the one-dimensionally evaluated optotypes. In one embodiment, presenting optotypes to assess visual acuity by using a one-dimensional approach and then switching to the modified approaches, which use a more than one-dimensional approach, is preferred because it allows for an accurate vision assessment in the most time-efficient manner. In an embodiment, the modified approach comprises a simple adaptive algorithm estimating a threshold along a single dimension (of stimulus size) changing to an acuity extrapolation algorithm that extrapolates acuity thresholds in a 2D space from multiple estimated acuity thresholds (each estimated by the aforementioned adaptive algorithm).

As described above, a head mounted vision device, such as described with reference to FIGS. 1A-1F, provides a controlled environment for vision testing. The processing unit of the HMVD is configured to control parameters of the visual environment such as a controlled testing distance (i.e., symbol size in degrees visual angle), background luminance level, contrast between the optotypes and their background, sequence in which the optotypes are presented, fixation cues, cues when a new optotype is presented and duration of the optotypes to be presented for recognition. In an embodiment, both cues that confirm to the user whether he/she made a response and any cue that tells the user whether he/she made a correct or incorrect response are also defined.

Controlling these variables results in a test that has the same measurement conditions for all users being tested, and for the same user providing the same measurement conditions at different times, thereby reducing variance and improving diagnostic results allowing for comparison between users and over time. The processing unit of the HMVD may be configured to generate visual or auditory cues to the user of the HMVD that are indicative of whether the user made a response that was successfully received by the processing unit of the HMVD, whether the user made a response that was accurate, as determined by the processing unit of the HMVD, and/or whether the user made a response that was inaccurate, as determined by the processing unit of the HMVD. The visual or auditory cues may be in any form, including emojis, icons, graphics, video, text, or auditory messages.

Vision testing using a head mounted vision device provides an objective testing of a user's left and right eyes without the user noticing which eye is being tested, thereby substantially eliminating subjective biases. Vision testing using a head mounted vision device also allows for the self-administration of vison tests with limited supervision at home or at a location where access to a shared device is available.

As previously described, however, a head mounted display presents some limitations, the primary being that only a limited amount of pixels are available to display the test image. As the display screen is in close proximity to a user's eyes and the pixel density of the device is limited, at smaller sizes, the limited resolution becomes more apparent and changes the optotypes shape from the standard shape. In one embodiment, the present specification provides systems and methods for assessing the visual acuity of users and compensating for the limited resolution of the head mounted display, by measuring users' contrast sensitivity (CS) and mapping the measured CS to acuity.

In another embodiment, an acuity threshold is extrapolated from multiple acuity thresholds measured at different fixed contrast levels, thereby not requiring contrast sensitivity measurement. In an embodiment, the present specification provides systems and methods for assessing the visual acuity of users and compensating for the limited resolution of the head mounted display, by measuring a user's visual acuity threshold, for a given size optotype, at different contrast levels, to generate multiple visual acuity thresholds and then extrapolating a visual acuity assessment from those multiple visual acuity thresholds. The present specification provides methods and systems for measuring visual acuity of users of a head mounted display and compensating for the limited resolution of the head mounted display by measuring the sensitivity of other visual parameters and mapping those other visual parameters to acuity.

To describe the improved visual acuity methods, a Landolt C visual acuity testing method will be described, although it should be appreciated than any visual acuity test that employs a multiple alternative forced choice (m-AFC) paradigm may be employed. In embodiments, the present specification provides a method of conducting vision tests such as, but not limited to: a distance visual acuity test, a contrast sensitivity test, and a contrast acuity by using the Landolt C (or Landolt ring) or any other m-AFC testing paradigm. In various embodiments, the device and method of the present specification provide for measurement of contrast sensitivity of a user, which may be used to extrapolate an acuity threshold of the user.

More specifically, the aforementioned vision tests are preferably structured as four alternative forced choices (4-AFC), which means that, when an optotype is presented to a user, there are 4 possible response alternatives that the user may select after evaluating the presented optotype, with precisely one response alternative defined as “correct”. For example, the four response alternatives may be “up”, “down”, “left” and “right”, corresponding to the four possible orientations of the Landolt C.

In an embodiment, before presentation of an optotype to the user, a signal such as, but not limited to an audio signal is transmitted to signal the onset of the Landolt C test, which is then presented at the center of a display screen of the head mounted display device being used to conduct the vision test, for approximately 1.5 seconds. The user may evaluate the optotype and provide his/her evaluation as a verbal response during this initial 1.5 seconds or during the subsequent 3.5 second period where the Landolt C is no longer presented. It should be appreciated that the response time period, during which a user is required to respond to the stimulus, the stimulus itself, or the periods between the stimuli, referred to as the inter-stimulus interval, may be modified and each could be used as a second or third analytical dimension, as further described below. For example, the stimulus duration can be reduced to a few milliseconds to display an optotype in a flash. In embodiments, the stimulus duration may be reduced to increase the visual challenge and test for higher acuity. In an embodiment, other than changing the stimulus duration, or subsequent response interval, a separate inter-stimulus interval (ISI) may be added in the periods between the stimuli.

If the user does not provide an acceptable response during the combined 5 second response period, the test is repeated. In other embodiments, the only other stimulus presented during the test is a fixation cue consisting of a circle with crosshairs that is strategically placed far enough away from the Landolt C to minimize crowding effects, which are decrements in detection performance due to interference from a nearby stimulus. The fixation cue is preferably displayed at all times. In embodiments, the Landolt C optotypes presented to the user during the vision tests are calibrated to International Organization for Standardization (ISO) standards, and background luminance during the presentation is set to 85 cd/m² which is within the range specified by ISO. In embodiments, the Landolt C optotypes are presented at a decrement contrast, i.e. at a darker shade of gray than the background on which the presentation is being displayed, wherein a contrast value of one represents the color black.

In embodiments, the first size visual acuity test, as described above, measures visual acuity of a user between 20/80 and 20/5000, or equivalently between 0.6 and 2.4 LogMAR. The lower limit of 20/80 may be a constraint imposed by the pixel size of the head mounted display device being used to present the test to the user, wherein given the distance between the user's eye and the display, the power of the lenses used, and the resolution of the display, the resolvable lower visual acuity bound corresponds to approximately 20/80, or a range of 20/70 to 20/90. More specifically, when using the Landolt C in a head mounted display device, a limiting factor is degrees visual angle for one pixel when viewing displayed images in the head mounted display. The Landolt C is defined to have gap and stroke widths that are ⅕ the diameter of the C. The smallest C that can therefore be presented, without anti-aliasing, is where the gap width equals 1 pixel. To measure 20/20 acuity, the gap width must be 1 minute of arc. A gap width shown in a conventional head-mounted vision device, such as with a ‘Samsung S8’ mobile phone fitted in a head mounted display, would equate to approximately 4.2 minutes of arc since the limiting factor for the Landolt C testing method is defined by a degrees visual angle when viewing the Landolt C gap in a display of the head mounted vision device. This corresponds to an equivalent acuity of approximately 20/84, necessitating an improved approach to determining visual acuity of 20/80 or better.

In an embodiment, the first size visual acuity test, as described above, is effectuated using different sized Landolt C presented at a contrast of 1 (the Landolt C is black) and, when the user's visual acuity is determined to be potentially better than 20/80, the second visual acuity test, further described below, is implemented, causing the contrast of the Landolt C to vary away from 1. In either case, the size of the Landolt C may vary between trials and is specified by an adaptive algorithm that estimates both a slope and thresholds at fixed contrast levels. In various embodiments, the present specification also provides an acuity extrapolation algorithm that works with any type of adaptive algorithm that can estimate an acuity threshold at fixed contrast level.

For example, the Landolt C stimulus size for conducting a distance visual acuity test is randomly chosen from within the 95% confidence interval (CI) of the likely distribution of the estimated visual acuity threshold derived from historically obtained responses to all previously conducted distance visual acuity tests while assuming a defined shape of a given psychometric function, such as Weibull. More specifically, the adaptive algorithm is initialized with a Landolt C optotype having a size of 20/960 and ends when the 95% CI is small enough to cross a predetermined threshold. In this case, an estimated visual acuity threshold is the maximum likelihood estimate given the responses to all presented stimuli during the test and represents 62.5% correct on the Weibull function. In various embodiments, the distance visual acuity test is estimated to take no more than 3 minutes.

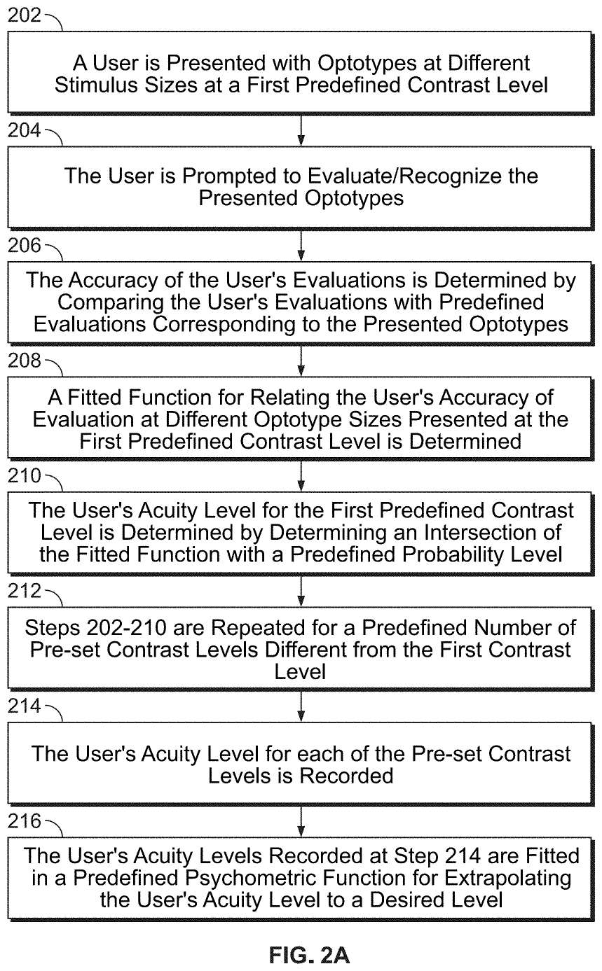

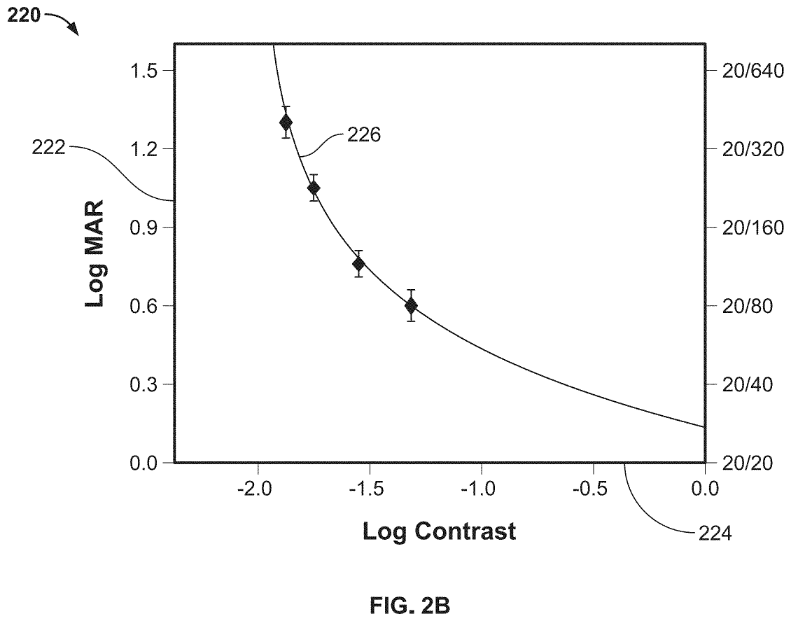

FIG. 2A is a flow chart illustrating a process of extrapolating a user's vision acuity level to a desired level by using a head mounted display system, in accordance with some embodiments of the present specification. At step 202, the head-mounted vision device generates, using its processor, memory, and a plurality of stored programmatic instructions, data representative of a plurality of optotypes which is shown in the display and therefore presented to a user. The optotypes comprise different sizes of symbols or letters at a first predefined contrast level or at a first predefined visual characteristic (such as color, brightness, hue, or saturation), as further described below. In embodiments, the optotypes represent a first plurality of visual stimuli. The first predefined contrast level or first predefined visual characteristic represents at least one of a first plurality of characteristics of the first plurality of visual stimuli. The visual stimuli may be presented in various forms. In some embodiments, the first plurality of visual stimuli is presented in a form of a grid defined by two or more vertical lines intersecting two or more horizontal lines. The grid covers a first plurality of coordinate locations in the user's visual field. In some embodiments, the grid covers the entirety of the user's visual field. In some embodiments, the grid is an Amsler grid. In some embodiments, the grid is defined by at least five vertical lines intersecting at least five horizontal lines to create equally sized boxes.

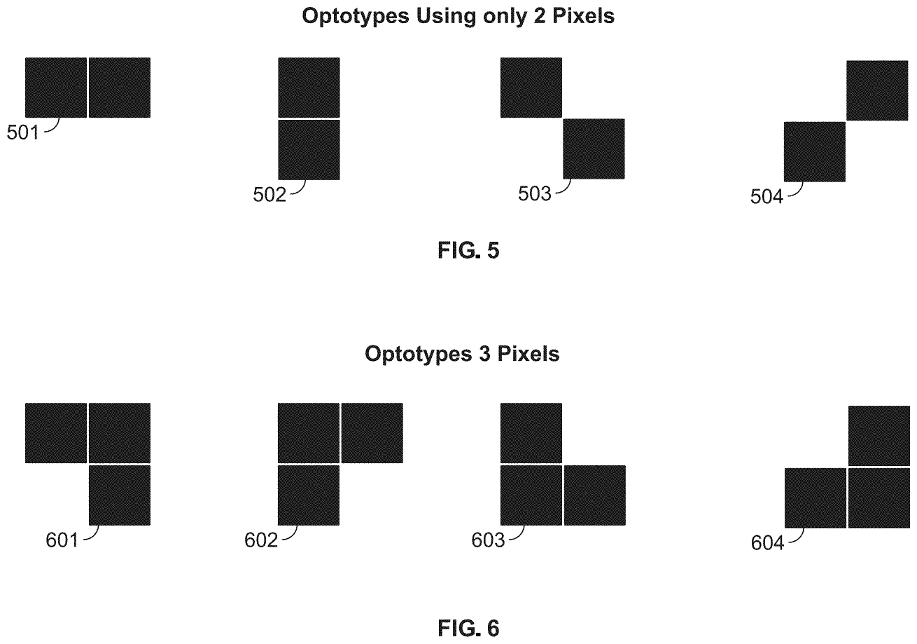

In embodiments, the presented optotypes are one of: Landolt ‘C’, tumbling ‘E’, or Sloan letters. In an embodiment, any kind of optotypes may be presented, such as, but not limited to, the optotypes that can be displayed with only a small number of pixels, for example, two pixels, as shown in FIG. 4, or three pixels, as shown in FIG. 5. The pixels may be presented adjacent to each other 501, 502, 601, 602, 603, 604 or in a diagonal relationship to each other 503, 504, 601, 602, 603, 604. These optotypes can be presented in smaller sizes on displays with limited resolution. In an embodiment, the method of the present specification applies a smoothing or softening filter to optotypes before they are presented to provide antialiasing causing the presented optotypes to be displayed with improved readability even with the limited number of pixels available on the display. In embodiments, antialiasing enables presentation of optotypes that do not strictly adhere to the optotype definition (such as, for example, a Landolt C with sharply defined edges), thus allowing optotypes to be presented at many more sizes than would be otherwise possible.

In an embodiment, the optotypes presented to the user are displayed with edges being blurred to further decrease the contrast in display with low contrast resolution display devices. Pixels that are on the corner and would be displayed half are represented with shades of gray wherein a darker shade of gray depicts more pixels that would have been presented if a fractional display were possible. In an embodiment the background luminance level of the optotypes presented to the user is changed, for causing an increase or decrease in contrast levels and bringing about a change in the 20/80 vision acuity threshold. In an embodiment, increment thresholds (white on gray) instead of decrement thresholds (black on gray) are used for simulating decreased contrast levels, as increment thresholds are higher than decrement thresholds.

In an embodiment, the optotypes presented to the user are displayed with dithering to decrease the contrast in displays having low contrast resolution as this allows for raising tested threshold values. Each display can provide a limited number gray or luminance levels only, such as 256. The discrete luminance variation between two levels is fixed. As a fine grain modification of luminance levels may be helpful in some embodiments, additional grey levels are simulated by dithering an optotype between two grey levels by used e.g. mixing two gray levels every other pixel in a checkerboard pattern. Other variations of proportions such as mixing 2 from one and 1 from the other are also applicable.

In various embodiments, the head mounted vision device, being used in the present specification for determining the user's visual acuity, provides a method for tracking the eye of the user to diagnose conditions that can affect the acuity measurement and resulting diagnosis of the user. A rear facing camera may be used for this purpose. The eye tracking also allows for verification of user attention and correct test procedure, to ensure that the user is looking at the presented optotype for evaluation. In various embodiments, the head mounted display device also provides a distance adjustment by measuring (optically or by other means) the distance from display to the user's eye and allows for compensation of distance between the user's pupils by adjusting a location (of display) and display size of the presented optotype accordingly.

At step 204 the user is prompted to evaluate/recognize the presented optotypes. In embodiments, the user may be prompted to submit his/her evaluation of the presented optotypes via audio or visual means by using a microphone, touch-screen, keyboard, joystick or remote control/wireless means, eye gaze, eye gestures, hand gestures, head gestures or a mix of said means by using the head mounted display device.

At step 206, the head mounted vision device determines the accuracy of the user's evaluations by comparing the user's evaluations with predefined evaluations corresponding to the presented optotypes. In some embodiments, determining the accuracy of the user's evaluations comprises detecting a discrepancy based on a comparison of the first plurality of characteristics with the user's response that is indicative of the visual characteristics of the first plurality of visual stimuli experienced by the user. In some embodiments, detecting the discrepancy is further achieved by receiving a response from the user, indicative of the visual characteristics of the first plurality of visual stimuli experienced by the user, and comparing the visual characteristics of the first plurality of visual stimuli experienced by the user with the first plurality of characteristics to identify the discrepancy. The detected discrepancy is stored as a first set of data. In embodiments, the discrepancy is indicative of one or more deficits in the visual field and comprises at least one of a partially missing vertical line, a partially missing horizontal line, a partially wavy vertical line, a partially wavy horizontal line, a partially blurred vertical line, or a partially blurred horizontal line. In some embodiments, the head mounted vision device associates first coordinate location from the first plurality of coordinate locations with the discrepancy and stores the detected discrepancy and first coordinate location as a first set of data.

In embodiments, the head mounted vision device accesses a database comprising accurate evaluation results corresponding to optotypes used for vision testing including the ones presented to the user, and compares the user submitted evaluation results with corresponding pre-stored results for determining the user's accuracy. In the multiple alternative forced choice paradigm (m-AFC), as described above, accuracy is determined from a given trial, or assessment session, by scoring the subject's response as either correct or incorrect and correlating the provided responses with a probability correct value assessed across many trials at the same stimulus size (for acuity) or same contrast level (for contrast sensitivity). It should be appreciated that the m-AFC encompasses a series of four choices where a viewer must select the right orientation of an optotype, as well as yes-no paradigms where a user may communicate whether he or she does (“yes”) or does not (“no”) see a given stimulus.

At step 208, the head mounted vision device fits a psychometric function that relates the user's accuracy of evaluation at different optotype sizes presented at the first predefined contrast level (or other pre-defined visual characteristic). At step 210, the head mounted vision device determines the user's acuity level for the first predefined contrast level (or other pre-defined visual characteristic) by intersecting the fitted function with a predefined probability level. This yields a first value representing a first estimate of visual acuity at a first contrast level and a plurality of optotype stimulus sizes.

At step 212 the steps 202-210 are repeated for a number of pre-defined contrast levels (or other pre-defined visual characteristic) different from the first contrast level (or the first pre-defined visual characteristic). For example, the user may be presented with optotypes at different stimulus sizes at a second, third, fourth . . . nth predefined contrast level, wherein ‘n’ is a predefined number. This yields a plurality of additional values representing a plurality of estimates of visual acuity at a plurality of contrast levels, each of the plurality of contrast levels having a plurality of optotype stimulus sizes associated therewith. In embodiments, the first set of data is used to generate and display a second plurality of visual stimuli. The second plurality of visual stimuli includes as least one of a second plurality of characteristics and is associated with a second plurality of coordinate locations. In embodiments, a number of the second plurality of coordinate locations is less than a number of the first plurality of coordinate locations. The user provides responses indicative of the visual characteristics of the second plurality of visual stimuli experienced by the user. In some embodiments, the second plurality of coordinate locations are only positioned at the first coordinate location. In embodiments, at least one of the second plurality of coordinate locations, a temporal frequency of the second plurality of visual stimuli, or the second plurality of characteristics of the second plurality of visual stimuli presented to a left eye of the user differs from at least one of the second plurality of coordinate locations, a temporal frequency of the second plurality of visual stimuli, or the second plurality of characteristics of the second plurality of visual stimuli presented to a right eye of the user. In some embodiments, the head mounted vision device is further configured to use the first set of data to determine a first area in the visual field having a determined detection below a predefined value and a second area in the visual field having a determined detection above a predefined value. The head mounted vision device is configured to set a temporal frequency of the second plurality of visual stimuli in the first area to be different from a temporal frequency of the second plurality of visual stimuli in the second area. In embodiments, the head mounted vision device is further configured to use the first set of data to determine a region that is smaller than the visual field of the user corresponding to an area of visual impairment. The head mounted vision device may then present the second plurality of visual stimuli only within the determined region.

At step 214, the head mounted vision device, or a server in data communication with the HMVD, stores the user's acuity level for each of the pre-set contrast levels (or other visual characteristics). At step 216, the head mounted vision device fits all of the user's acuity levels recorded at step 214 with a predefined psychometric function to determine a final value indicative of the user's acuity level. In embodiments, step 216 comprises replacing the threshold parameters in a psychometric model with the obtained acuity relations. The head mounted vision device determines attributes of the user's visual field based on the detected discrepancy and the responses that are indicative of the visual characteristics of the second plurality of visual stimuli experienced by the user. In embodiments, the head mounted vision device is configured to use the determined attributes to identify one or more regions of visual impairment in the visual field of the user. The head mounted vision device may generate a display which visually overlays the identified region or regions of visual impairment onto the user's visual field. In an embodiment, the head mounted vision device uses the psychometric model disclosed by Alexander et al. in the paper titled “Visual Acuity and Contrast Sensitivity for Individual Sloan Letters” published in ‘Vision Research’ Volume 37, Issue 6, March 1997, Pages 813-819, which is incorporated herein by reference, is used at step 216 by replacing the contrast sensitivity thresholds by the acuity values recorded at step 214.

In some embodiments, the data representative of the plurality of optotypes at the first predefined visual characteristic is generated with reference to a first session of evaluation of the user's vision acuity level. The data or additional values at the plurality of contrast levels, each of the plurality of contrast levels having a plurality of optotype stimulus sizes, is generated with reference to a first session of evaluation of the user's vision acuity level. In some embodiments, the first and second sessions occur at different points in time to verify or check disease progression based on past measurements (related to, for example, the first session).

In an embodiment, the present specification provides a mathematical mapping function that fits a predefined psychometric function such as Alexander's model. In an embodiment, the mathematical function is a four parameter model of the probability of a user correctly evaluating a stimulus at any point in a two dimensional (2D) space where on one axis the size of the stimulus is being varied and on the other axis the contrast is being varied. In other embodiments a different number of parameters is used. In embodiments, the present specification provides an adaptive algorithm over the 2D space for extrapolating the user's visual acuity to a desired level. The adaptive algorithm/mapping function is developed by using prior published information about the user's acuity relations, estimates from clinical tests for specific optotypes with gradual adjustment for refinement, estimates from results of other users of the device and method of the present specification that are subjected to optotypes to be tested after their visual acuity has been established. FIG. 2B is a graph illustrating the relationship between stimulus size and contrast, in accordance with an embodiment of the present specification. Graph 220 plots variance in stimulus size marked on y-axis 222 against variance in contrast values marked on x-axis 224. In an embodiment, plot 226 represents the 2D space which relates variance in stimulus size to variance in contrast values, thereby enabling relating the measured relationship (from the user) to a known curve which is indicative of specific vision levels. In an embodiment, plot 226 represents a 2D acuity extrapolation figure for a person with 20/30 vision.

In an embodiment, mapping functions are optimized for faster tests, reduced user fatigue and increased efficiency by estimating the threshold parameters of the function from: prior probability distribution from average population overall or in the relevant subgroup of age and gender, wherein Bayesian inference is used to update probabilities; visual measurements conducted in different locations; historic data of vision tests conducted on the same user previously; and historic data that determined existing vision conditions in the same user.

In an embodiment, the present specification provides a method of extrapolating visual acuity of a user on multiple different head mounted display devices by compensating for different characteristics of said devices. For example, the present specification provides a method of extrapolating visual acuity of a user with the same accuracy over multiple different head mounted display devices using different types of mobile phones. Hence, the present specification provides a method of calibrating different head mounted vision devices to provide the same accuracy level when being used for extrapolating visual acuity of a user. The calibration may be applied automatically by identifying hardware properties of a head mounted display device or manually configured, such as, but not limited to: non-square pixels (displays for which the resolution is not proportional to the number of pixels in width and height); different contrast levels (luminance resolution); display technology such as, but not limited to LCD, OLED, AMOLED, Super AMOLED, TFT, IPS; colors of pixels (RGB composition); edge sharpness, i.e. contrast between pixels; different luminance differences for different gray levels; and Gamma values for display device.

As would be apparent to persons of skill in the art the principle of acuity extrapolation through replacing the threshold parameter in typical psychometric functions with a parameterized relation among acuity thresholds may be applied to any psychometric model that relates acuity thresholds as a function of any physical property of the stimulus provided for visual testing. For example, in embodiments psychometric models in which the threshold relation may be provided for different stimulus durations or temporal profiles (e.g., a square wave, a Gaussian, or a raised cosine), or for different background luminance levels may be used at step 216. In an embodiment, psychometric models in which the thresholds are increment or decrement thresholds for any optotype is used at step 216. In other embodiments other psychometric functions developed for measuring vision acuity may also be used.

In various embodiments, the process of determining a user's visual acuity by using a head mounted display system may be carried out with fixing a contrast level and varying other parameters such as but not limited to a perceived, apparent or virtual distance of the user from the presented optotype. As described above, any visual characteristic of the stimulus/optotype, or any characteristic of how the stimulus/optotype is displayed, may be altered and used to generate data for the multi-dimensional adaptive algorithm, as described herein, to find acuity threshold relations that may be used for an improved determination of visual acuity. Such characteristics include, but are not limited to, color, background luminance, temporal profiles, optotype shapes, white on gray (increment) or black on gray (decrement) thresholds, response time periods, and/or inter-stimulus interval time periods. For example, by varying background luminance (in place of varying contrast, as described above), one can increase or decrease the degree of visually perceived contrast and achieve a similar two dimensional analytical space, as described above. As another example, the system may implement increment thresholds (white on gray) as opposed to decrement thresholds (black on gray) to simulate decreased contrast since increment thresholds tend to be slightly higher than decrement thresholds.

Similarly in various embodiments other visual parameters, other than visual acuity, can be assessed and quantified from a variety of different stimuli, such as the ability to perceive color, motion, contrast, peripheral input, adjustment to light from stimuli such as but not limited to color, background luminance, temporal profiles, optotypes, and white on gray (increment) or black on gray (decrement) thresholds.

As previously described, it should be appreciated that the head mounted visual assist device implements a contrast acuity test to estimate visual acuity up to a predefined threshold, such as greater than 20/80, and up to 20/20 or better. In embodiments, the contrast acuity test is only employed if the distance visual acuity test estimates an acuity threshold of better than 20/80 (or a range of 20/70 to 20/90), which means that the estimated threshold is unreliable. The contrast acuity test estimates acuity between 20/20 and 20/80 by leveraging an empirically determined relation among acuity thresholds, which is a multi-parameter exponential known to provide a good fit to acuity thresholds estimated from Landolt C presented at different contrast levels (each acuity threshold is estimated for a different contrast level). In an embodiment, acuity threshold between 20/20 and 20/80 is extrapolated using this multi-parameter model from 3-10 acuity thresholds at different contrast levels (the number of estimated thresholds depends on the test subject's responses). At each contrast level, 3-4 optotypes are first presented to the user to test whether the acuity threshold lies between 20/80 and 20/960 or if the acuity threshold is greater than 20/80. If the range is estimated to be between 20/80 and 20/960, the distance visual acuity test is used to estimate the threshold; otherwise, no threshold is estimated. This procedure reduces the overall time for the contrast acuity test to a maximum of 10 minutes.

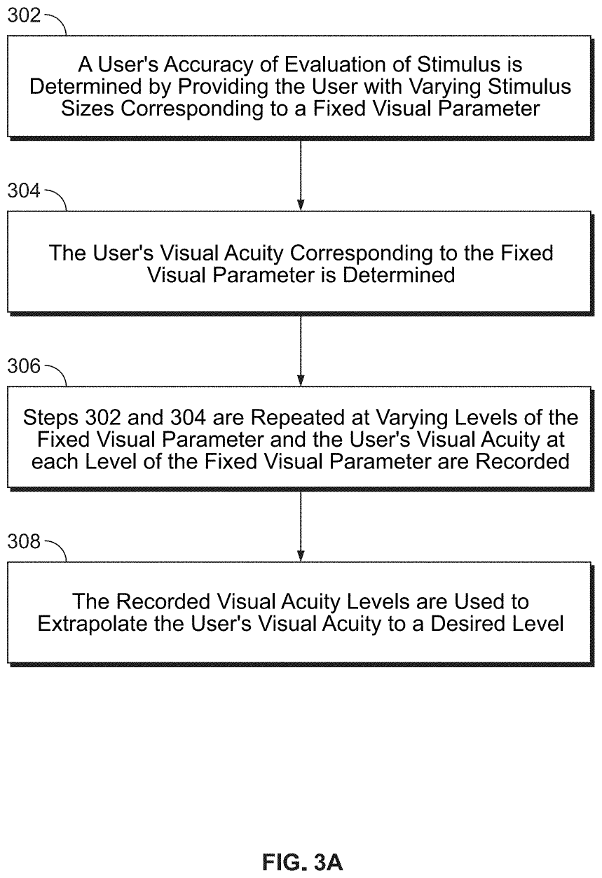

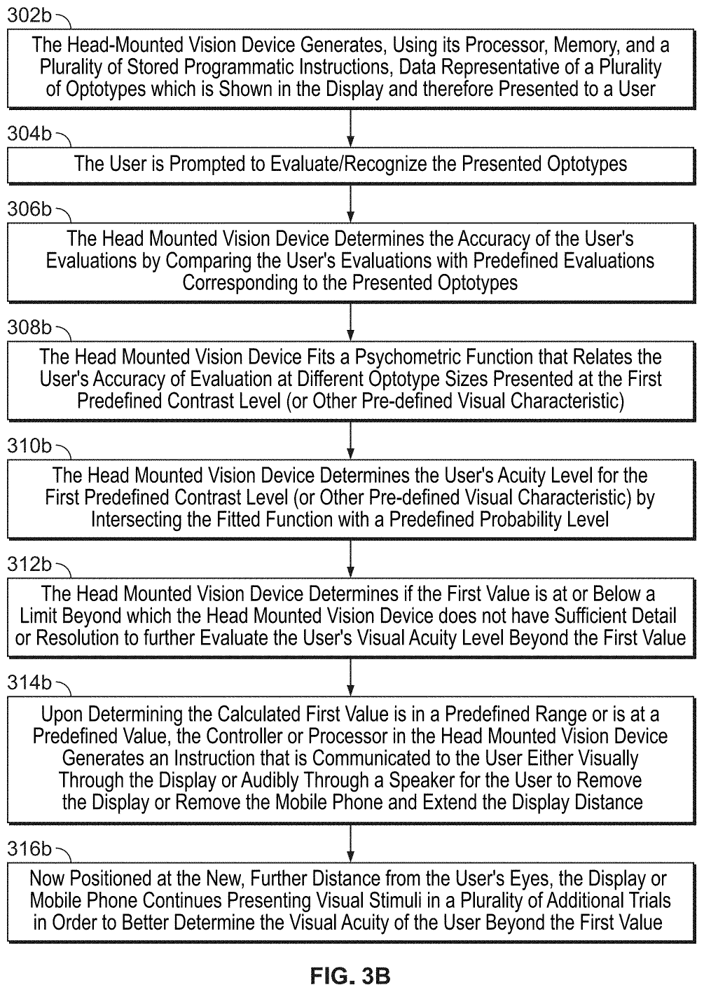

FIG. 3A is a flow chart illustrating a process of extrapolating visual acuity by using a head mounted vision device, in accordance with some other embodiments of the present specification. At step 302a a user's accuracy of evaluation of stimulus is determined by providing the user with varying stimulus sizes corresponding to a predefined/fixed visual parameter. In embodiments, the user may be presented with stimulus in the form of optotypes at different stimulus sizes at a first predefined contrast level by using the head mounted display device (described above) for evaluation. The user may be prompted to evaluate/recognize the presented optotypes and the user's accuracy of evaluation may be determined by comparing the user's evaluations with predefined evaluations corresponding to the presented optotypes. At step 304a, the user's visual acuity corresponding to the fixed visual parameter is determined. In an embodiment, firstly a fitted function for relating the user's accuracy of evaluation at different optotype sizes presented at the first predefined contrast level is determined and then the user's acuity level for the first predefined contrast level is determined by determining an intersection of the fitted function with a predefined probability level. In other embodiments, various other methods of determining the user's visual acuity corresponding to the fixed visual parameter may be used. At step 306a, the steps 302a and 304a are repeated at varying levels of the fixed visual parameter and the user's visual acuity at each level of the fixed visual parameter are recorded. At step 308a, the recorded visual acuity levels are used to extrapolate the user's visual acuity to a desired level. In an embodiment the recorded visual acuity levels are fitted in a known psychometric function for determining the user's visual acuity down to at least 20/20 level.

FIG. 3B is a flow chart illustrating another process of determining a user's vision acuity level to a desired level by using a head mounted display system. At step 302b, the head-mounted vision device generates, using its processor, memory, and a plurality of stored programmatic instructions, data representative of a plurality of optotypes which is shown in the display and therefore presented to a user. The optotypes comprise different sizes of symbols or letters at a first predefined contrast level or at a first predefined visual characteristic (such as color, brightness, hue, or saturation), as further described below. In embodiments, the presented optotypes are one of: Landolt ‘C’, tumbling ‘E’, or Sloan letters. In an embodiment, any kind of optotypes may be presented, such as, but not limited to, the optotypes that can be displayed with only a small number of pixels, for example, two pixels, as shown in FIG. 4, or three pixels, as shown in FIG. 5. The pixels may be presented adjacent to each other 501, 502, 601, 602, 603, 604 or in a diagonal relationship to each other 503, 504, 601, 602, 603, 604. These optotypes can be presented in smaller sizes on displays with limited resolution. In an embodiment, the method of the present specification applies a smoothing or softening filter to optotypes before they are presented to provide antialiasing causing the presented optotypes to be displayed with improved readability even with the limited number of pixels available on the display. In embodiments, antialiasing enables presentation of optotypes that do not strictly adhere to the optotype definition (such as, for example, a Landolt C with sharply defined edges), thus allowing optotypes to be presented at many more sizes than would be otherwise possible.

In an embodiment, the optotypes presented to the user are displayed with edges being blurred to further decrease the contrast in display with low contrast resolution display devices. Pixels that are on the corner and would be displayed half are represented with shades of gray wherein a darker shade of gray depicts more pixels that would have been presented if a fractional display were possible. In an embodiment the background luminance level of the optotypes presented to the user is changed, for causing an increase or decrease in contrast levels and bringing about a change in the 20/80 vision acuity threshold. In an embodiment, increment thresholds (white on gray) instead of decrement thresholds (black on gray) are used for simulating decreased contrast levels, as increment thresholds are higher than decrement thresholds. In an embodiment, the optotypes presented to the user are displayed with dithering to decrease the contrast in displays having low contrast resolution as this allows for raising tested threshold values. Each display can provide a limited number gray or luminance levels only, such as 256. The discrete luminance variation between two levels is fixed. As a fine grain modification of luminance levels may be helpful in some embodiments, additional grey levels are simulated by dithering an optotype between two grey levels by used e.g. mixing two gray levels every other pixel in a checkerboard pattern. Other variations of proportions such as mixing 2 from one and 1 from the other are also applicable.

At step 304b the user is prompted to evaluate/recognize the presented optotypes. In embodiments, the user may be prompted to submit his/her evaluation of the presented optotypes via audio or visual means by using a microphone, touch-screen, keyboard, joystick or remote control/wireless means, eye gaze, eye gestures, hand gestures, head gestures or a mix of said means by using the head mounted display device.

At step 306b, the head mounted vision device determines the accuracy of the user's evaluations by comparing the user's evaluations with predefined evaluations corresponding to the presented optotypes. In embodiments, the head mounted vision device accesses a database comprising accurate evaluation results corresponding to optotypes used for vision testing including the ones presented to the user, and compares the user submitted evaluation results with corresponding pre-stored results for determining the user's accuracy. In the multiple alternative forced choice paradigm (m-AFC), as described above, accuracy is determined from a given trial, or assessment session, by scoring the subject's response as either correct or incorrect and correlating the provided responses with a probability correct value assessed across many trials at the same stimulus size (for acuity) or same contrast level (for contrast sensitivity). It should be appreciated that the m-AFC encompasses a series of four choices where a viewer must select the right orientation of an optotype, as well as yes-no paradigms where a user may communicate whether he or she does (“yes”) or does not (“no”) see a given stimulus.

At step 308b, the head mounted vision device fits a psychometric function that relates the user's accuracy of evaluation at different optotype sizes presented at the first predefined contrast level (or other pre-defined visual characteristic). At step 310b, the head mounted vision device determines the user's acuity level for the first predefined contrast level (or other pre-defined visual characteristic) by intersecting the fitted function with a predefined probability level. This yields a first value representing a first estimate of visual acuity at a first contrast level and a plurality of optotype stimulus sizes.

At step 312b, the head mounted vision device determines if the first value is at or below a limit beyond which the head mounted vision device does not have sufficient detail or resolution to further evaluate the user's visual acuity level beyond the first value. For example, if the first value is in a range of 20/70 to 20/90, the resolution of the display associated with the head mounted vision device may not be sufficient to further refine the estimate of the user's visual acuity. Accordingly, upon determining the calculated first value is in a predefined range (such as 20/70 to 20/90) or is at a predefined value, such as 20/80, the controller or processor in the head mounted vision device generates an instruction that is communicated to the user either visually through the display or audibly through a speaker in step 314b. The instruction informs the user that he or she must remove the display associated with the head mounted vision device, or more specifically, remove the mobile phone, and extend the display a distance from the user's eyes such that the testing distance increases relative to the prior trials. In one embodiment, the distance is roughly equivalent to the average arm length of users. In another embodiment, the distance is a set number of inches. In another embodiment, the distance is variable based on the user and is determined by the mobile device by a) capturing an image of the user and extrapolating a distance based on an average interpupillary distance or b) capturing an image of the user having a sizing object (such as a ruler) positioned proximate one or more portions of his or her face. At step 316b, now positioned at the new, further distance from the user's eyes, the display or mobile phone continues presenting visual stimuli in a plurality of additional trials in order to better determine the visual acuity of the user beyond the first value. Preferably, the visual stimuli have been adjusted based on the determined distance.

In an embodiment, the method of the present specification may be used to predict vision impairments by using tools such as statistical analysis and observed correlation between a user's performance in vision tests and vision impairments. In embodiments, data obtained by conducting vision tests by using the method of the present specification is particularly reliable as the controlled environment of said tests ensures low variance between tests and the use of a personal head mounted display device ensures that the test is performed on the same user in the same manner with directly transferable results. In addition, the consistency of test not only for the same user over time but also for different users allows to collect and evaluate the data from vision tests to find correlations between vision tests and later diagnosed vision impairments that may not be known at the time of testing. Data mining can be used explore the sets of data from recorded tests identify correlation between future vision impairments and features such as speed to identification, preference for certain optotypes, eye movement during before and after presentation with the optotype and other.

In embodiments, the head mounted display device described in the present specification may be used to conduct other vision tests such as, but not limited to an Amsler grid, which is a high contrast supra-threshold test and Humphrey visual field test, for diagnosing color vision or field of vision of a user.

It should be appreciated that the head mounted visual assist device may also implement a contrast sensitivity test that varies the contrast of the Landolt C from 0 to 1 (e.g. from 85 cd/m² down to 0 cd/m²) with the size of the Landolt C fixed at 20/800. In an embodiment, the contrast sensitivity test uses the same adaptive algorithm as described above with reference to the distance visual acuity test to determine the contrast level of the next stimulus/optotype presented to the user undertaking the test. More specifically, a final estimated contrast sensitivity threshold is the maximum likelihood estimate given the responses to all presented stimuli during the test given a predefined psychometric function, such as a Weibull, cumulative normal, logistic, or any other sigmoidal function. In an embodiment, the estimated threshold corresponds to 62.5% correct evaluations of the presented optotype submitted by the user. The algorithm is initialized with a Landolt C at contrast level being equal to 1. In various embodiments, the contrast sensitivity test is estimated to take no more than 3 minutes.

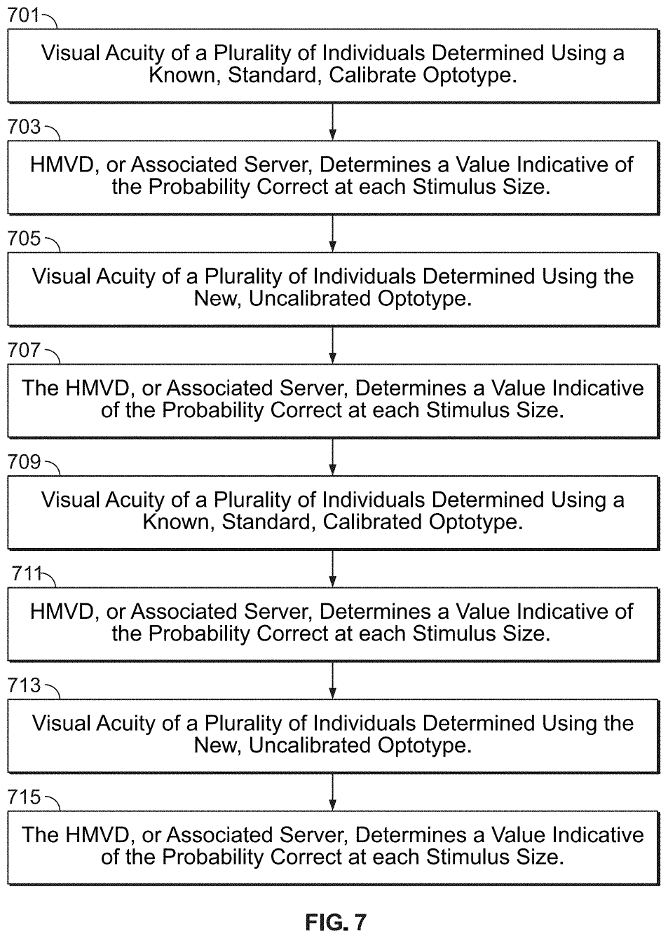

It should further be appreciated that, instead of implementing the two-dimensional adaptive algorithm described above in relation to the second visual acuity test, a new optotype may be developed, calibrated relative to standard optotypes, and used in place of standard optotypes in a HMVD. Referring to FIG. 7, in one embodiment, the visual acuity of a plurality of individuals is determined 701 using a known, standard, calibrated optotype, such as Landolt C, which is presented at different optotype stimulus sizes in the HMVD. The HMVD, or associated server, determines 703 a value indicative of the probability correct at each stimulus size. Using the HMVD, the process is repeated for a new optotype having improved display resolution, thereby allowing for an accurate assessment of a viewer's visual acuity below 20/80, and preferably to at least 20/20. More specifically, the visual acuity of a plurality of individuals is determined 705 using the new, uncalibrated optotype, which is presented at different optotype stimulus sizes in the HMVD. The HMVD, or associated server, determines 707 a value indicative of the probability correct at each stimulus size.

The HMVD, or associated server, fits 709 a psychometric function to the data generated from the known, calibrated optotype, which determines the probability correct at each point on a first axis, e.g. the x-axis or size for the known, calibrated optotype. The HMVD, or associated server, determines 711 a point on a second axis, e.g. the y axis, corresponding to a measured probability correct for the new, uncalibrated optotype, thereby allowing the HMVD, or associated server 713 to determine a corresponding point on the first axis and, therefore, a size of the known calibrated optotype equivalent to the measured, new optotype. After calibrating a plurality of such sizes for the new optotype over a sufficiently large population size, the HMVD or associated server then performs the first visual acuity test, as described above, using the new, now calibrated, optotype and applying a one dimensional adaptive algorithm which assesses the probability correct at different optotype sizes. As such, the HMVD, or associated server uses 715 the new optotype in a one dimensional adaptive algorithm to assess the visual acuity of a user. The calibration approach may be applied using any of the aforementioned visual, or display, characteristics described herein and is not just limited to the size of the optotype.

In an embodiment, the present specification enables users to provide key diagnostic data to a clinician asynchronously. For example, a user conducting a vision test by using the head-mounted vision device (HMVD) of the present specification, may record data related to his/her vision as well as data related to the device and the environment in which the device is operating, and submit the recorded data to a clinician for review at a later time.

The clinician may then record feedback as well as 1) comments specifying any parameter settings that may need to be changed and 2) content that should be displayed on the user's headset screen. The clinician may submit the recorded feedback and comments to be viewed by the user at a later time.

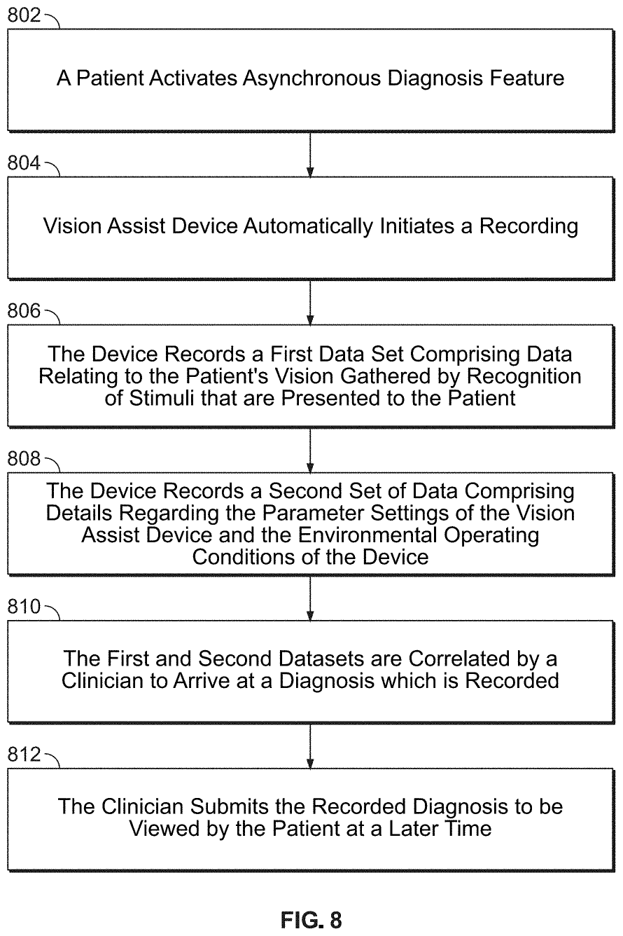

In embodiments, the device of the present specification, when operating in asynchronous mode, may also be used to administer tests, such as visual acuity and visual field tests. FIG. 8 is a flowchart illustrating exemplary steps of executing an asynchronous diagnosis feature in the vision assist device, in accordance with an embodiment of the present specification. In an embodiment, when the user activates this feature at step 802, the vision assist device of the present specification automatically initiates a recording at step 804. The activation may occur by the user selecting a specific icon in one or more graphical user interfaces. The activation may further occur by the user responding to a notification, prompt, message, communication, or other data generated by the clinician and indicative of the clinician recommending the user independently engage in one or more vision tests without the clinician's real-time guidance or observation.

Regardless of how it is activated, during the recording, settings from the device as well from the environment in which the device is operating are recorded. In embodiments, the recorded data comprises two datasets, wherein a first dataset is directly indicative of the user's vision and a second dataset is indicative of the reliability and accuracy of the first dataset and comprises information regarding the environment in which the vision assist device is operating. The first data set comprises data relating to the user's vision gathered by recognition of stimuli that are presented to the user and is recorded at step 806. In embodiments, the second set of data comprises details regarding the parameter settings of the vision assist device and the environmental operating conditions of the device and is recorded at step 808. In an embodiment, parameter settings of the headset device comprise parameter settings related to screen recording, audio from the device and from the user (speaker and microphone settings), screen brightness, contrast, and filter configuration of filters that improve vision. In an embodiment, parameter settings related to environmental operating conditions of the headset device comprise parameter settings related to time of the day, brightness of the environment, temperature, humidity, and indoor/outdoor operation.

In embodiments, the first and second datasets are compared at step 810 by a clinician to arrive at a diagnosis which is then recorded. The clinician submits the recorded diagnosis so that it can be viewed by the user at a later time, at step 812.

In an embodiment, the first and second dataset parameters may be recorded together or simultaneously. In an embodiment, when the vision assist device records the first dataset and concurrently captures the second dataset, the first dataset is modified based on the captured second dataset. In some embodiments, the modification is continual or dynamic and includes normalization of the first dataset relative to a standardized second dataset. In another embodiment, the parameters may be recorded independently in scenarios that are not aimed at testing vision but at improving vision, for example when the headset device is being used with regular filters to improve vision or to determine the functional parameters of the vision assist device. In embodiments, the second dataset is primarily used for determining the conditions the vision assist device is operating in. In an embodiment, the clinician reviews the second dataset to understand how to interpret the first dataset. In some embodiments, the clinician reviews and interprets the first dataset, in the context of the second dataset, prior to arriving at a diagnosis.

In accordance with some aspects of the present specification, the system provides reliability data to enable a clinician to determine an extent to which a user's vision test data may be trusted and therefore whether or not the vision test needs to be performed again. In various embodiments, reliability of a vision test is determined based on a plurality of factors or metrics such as a) false positive percentage, b) false negative percentage, c) fixation loss and d) mean deviation which is reflective of an amount by which the user's sensitivity deviates. For example, if the user's test data indicates 15% fixation loss and 20% false positive, then the test data is construed to be unreliable. In some embodiments, the vision-assist application (executed on the smartphone) is configured to perform a reliability analysis using one or more of the plurality of factors or metrics, in the context of the user's vision test data, in order to determine and recommend to the clinician whether the test data is reliable or not.

In some embodiments, the vision-assist application is further configured to calibrate the reliability analysis based on: a) monitoring the luminescence or brightness level of the smartphone or b) identification of the type of smartphone that, in turn, indicates an expected level of luminescence. It should be noted that while the vision-assist application is configured to control the luminescence level of the smartphone, the application still does not know if the smartphone is outputting the desired or required luminescence level. Therefore, in embodiments, the application needs to monitor luminance and actually calibrate the reliability analysis or determine the type of smartphone being used and calibrate the reliability analysis based on the type of smartphone.

In order to calibrate based on the type of smartphone, the vision-assist application is configured to either actively get or acquire information on the type of smartphone or automatically detect the type of smartphone. In some embodiments, once the type of smartphone is determined, the vision-assist application loads a calibration file and then uses that calibration file to set the brightness for each of 256 gray levels. In some embodiments, the calibration file could be, for example, a file having a data structure with a first column including 0 to 255 numerals (each indicative of a gray level) and a second column including luminescence level (in cd/m²) corresponding to each gray level in the first column. In practice, if the vision-assist application is displaying an Amsler Grid test, standards require using a luminescence level of 10 cd/m². Therefore, in embodiments, the vision-assist application is configured to find the closest luminance level and set the background and stimulus gray scale accordingly. The same is done for other vision tests (such as, for example, VFT).

In some embodiments, to ensure that luminance/brightness of display on the smartphone is accurate, the vision-assist application is configured to a) automatically shut off or prompt the user to shut off any restrictions on brightness (that is, low power mode, for example) and b) monitor for low battery of the smartphone. If the vision-assist application determines that the battery is low, the vision-assist application is configured to inform the user to charge the smartphone to a point sufficient that a vision test can be reliably conducted.

In some embodiments, the second dataset may also be mined for determining new correlations between the operating condition of the device and user vision. For example, by correlating the first dataset and second dataset, it is possible to determine the effects of time or temperature on user vision. In an embodiment, a statistical analysis of the comparison between the different values of the first dataset and second dataset is performed to determine dependencies (and is some cases, hidden dependencies) as mentioned above. In some embodiments, other methods of analysis such as, but not limited to regression techniques, for example, linear or polynomial regression for continuous data, and logistic regression for categorical data may be used. This may allow the clinician to more accurately interact with a user, notwithstanding distance and time differences, by experiencing or understanding more of the environment the user is exposed to, even absent an in-person assessment.

In another embodiment, neural network/AI is applied to at least one of the first dataset and the second dataset to determine at least one relationship between vision test results and the environment or applied filters. This may provide insight that a certain condition worsens (for one user or in general) during certain environmental conditions (such as, but not limited to, time of day, higher temperatures, and lower light conditions).

In an embodiment, the present specification also provides a video diary that allows a user to record his/her thoughts or observations during use of the vision assist device. The video diary enables users to share difficult or impaired situations with the clinician, via a narration of what the user is seeing at that time. In an embodiment, users may be prompted to record or a recording at certain times of the day or in specific situations may be automatically scheduled.

Further, in embodiments, the vision assist device of the present specification allows the user (or clinician) to share all of the acquired data with other providers for additional opinions.

In an embodiment, the vision assist device provides a photo-biomodulation treatment modality. As is known in the art, photo-biomodulation therapy involves application of red and near infra-red light to promote soft tissue healing, reduce inflammation and give relief for both acute and chronic pain. In an embodiment, the vision assist device comprises a plurality of light emitting diodes (LEDS) integrated into the device, at predefined locations, such that, upon activation, the LEDS direct light into a user's skin. In various embodiments, different treatment doses of said light are predefined corresponding to the different modes of operation of the vision assist device.

In an embodiment, a user may use gesture and voice to control the operation of the vision assist device of the present specification. In embodiments, a plurality of voice commands and eye/hand gestures may be predefined to be interpreted in different ways by the device, corresponding to different user vision profiles and the different modes of operation of the device. In an embodiment, a set of voice commands and/or gestures may be predefined via which users may use the vision assist device to administer a test, interact with clinicians, or adjust settings on a device filter.

In an embodiment, the vision assist device of the present specification provides a ‘user-assist’ feature for empowering friends/family of a user to help the user use the device beneficially. This feature is particularly useful where an elderly user is using the device and his/her adult child wants to help his/her parent.

In an embodiment, the vision assist device allows one or more predefined family member(s)/friend(s) of a user to create a script for guiding the user to use one or more features of the device. In an embodiment, the vision assist device provides the predefined family member/friend with a plurality of prompts based upon observed behavior of said family member/friend for creating the script. In an embodiment, the device observes the interactions between the family member/friend and the user by either using human or artificial intelligence to derive a structured script.

The device's observation may be used to improve the workflow of the script and may also be used to create bots for virtual trainers, individualized automatically generated training and assisted navigation. In an embodiment, the system of the vision assist device can also learn and adapt so that it can suggest next steps for treatment or diagnosis. The system may learn by using inputs from the system on the successful suggestions and treatments as feedback by future use and direct user interaction with subjective input on the quality of the guidance from the system. In another embodiment, a script may be created to automatically configure the device based on a known existing eye condition of the user. In an embodiment, the ‘user-assist’ feature may be implemented by combining a voice over IP phone call with screen sharing. In another embodiment, the ‘user-assist’ feature may be implemented by enabling the family member/friend to configure a vision assist device remotely. Configuration may comprise configuration of device parameters such as, but not limited to, audio volume, screen brightness, and filter configuration.

The present specification recognizes that insurance companies and/or government reimbursement programs may require remote monitoring of the user to happen at a predefined frequency or schedule, such as, for example, 5 to 20 days a month, in order to qualify for reimbursement. In some embodiments, the system 100 (FIG. 1A) generates prompts for the user at the predefined frequency or schedule in order to ensure that the user meets a required testing paradigm to qualify for the reimbursement. In some embodiments, the prompts are displayed to the user on his smartphone 110. In embodiments, the user's interaction with or response to the prompt may cause activation of the asynchronous diagnosis feature in the vision assist device in accordance with the steps of FIG. 8, for example. The activation may occur by the user selecting a specific icon in one or more graphical user interfaces. The activation may further occur by the user responding to a notification, message, communication, or other data recommending the user to independently engage in one or more vision tests without a clinician's real-time guidance or observation.

In some embodiments, if the user fails to comply and complete the tests, in accordance with the predefined schedule and prompts, the system 100 is configured to execute a predefined escalation process. In various embodiments, the predefined escalation process may entail one or any combination of: generating and communicating notices to a clinician (which may or may not be the same clinician who is already engaged with and treating the user), generating and displaying visual non-compliance warnings to the user, or generating and providing audio based non-compliance warnings to the user. In some embodiments, the clinician and/or warning notices may offer to reschedule the testing schedule and/or elicit feedback from the user regarding his reasons for non-compliance.

In an embodiment, the ‘user-assist’ feature comprises testing a user via a software module of the vision assist device for determining if the user has understood one or more modes of the device. In an embodiment, a user may be trained to perform functions such as, but not limited to, connecting the device to WiFi, using voice command to interact with the device, interacting with the device using buttons and/or swipe pad. In an embodiment, the software monitors the user while the user performs said functions and if the user makes a mistake while performing a function, the user is provided with a highlighted animation of the area of the vision assist device that performs that function, thereby enabling the user to verify said function.

In an embodiment, the vision assist device enables a user to perform daily living activities such as, but not limited to, reading a book, reading a prescription, and watching television. In embodiments, the device comprises object identification AI based software in order to prompt a user and provide assistance in performing said activities.

In an embodiment, the vision assist device uses a bot to provide a bot-scripted guide explaining to a user how to use specific features of the device. In embodiments, the bot may present the user with a plurality of image options and prompt the user to recognize the presented images. The bot may then create the script for guiding the user to use specific features (such as, but not limited to, selection of a filter) by using the user's responses.

In an embodiment, the present specification provides a method of automatically looping in predefined family members/friends of a user on a user consult call, wherein the call may be a scheduled call or an emergency call. In an embodiment, the vision assist device is coupled with an accelerometer to determine if a user using said device has slipped or fallen. In another embodiment, the vision assist device uses user audio and/or video images to monitor any rapid changes in said audio/video to determine if the user is in an emergency situation. In an embodiment, the vision assist device may scan the user's room's spatial orientation by using sensors such as, but not limited to LIDAR sensors, to determine if the user is in an emergency situation. In case the user is in an emergency situation (such as, but not limited to slipping and falling) the vision assist device automatically places a call to a clinician and causes predefined family members/friends to be looped in on the call.

In some embodiments, the head-mounted vision device (HMVD) may be used for conducting visual field and peripheral vision tests such as, but not limited to Humphrey VFT and Amsler grid tests. The HMVD provides a controlled environment for conducting the tests (where both luminance and visual angle degrees are controlled), thereby yielding improved accuracy compared to the tests being done without the use of HMVD.

In various embodiments, by using the HMVD the test environment may be controlled by controlling parameters such as ambient lighting and distance of the user's eye from the test screen. Further advantages of using the HMVD of the present specification to conduct VFT and Amsler grid vision tests include, but are not limited to, testing different eyes without the user noticing, tracking the user's eye by varying the test stimulus based on the user's eye focus, and verifying the user's eye fixation by using a blind spot and ensuring that the user cannot see the blind spot.

In some embodiments, the lens of an HMVD may introduce distortion in the testing results. However, the same can be accounted for and compensated with test location as presented, for example, by moving lenses to compensate for inter-pupillary distance, thereby allowing for calibration. A common HMVD calibration may be achieved by aligning squares presented to the left and right eye of the user during the test. While conducting said vision tests by using an HMVD, feedback may be received from the users via gestures, voice recognition and/or a handheld joystick/clicker.

The use of HMVD for conducting said tests enables a doctor to observe the user's eye, the stimuli provided, and the response via a recording of the test session. The doctor may also present the user with variations in the vision test to obtain improved test results or may conduct randomized trials to find the best suited test parameters for the user. In embodiments, the test stimuli may be varied to shapes such as, but not limited to disc, dot or square; fixation targets may be designed to attract the user's attention with a flash, crosshairs or a cross; and different filters may be applied to test their suitability to compensate for vision impairment.

In various embodiments, an HMVD may be used to register, collect, store, process and transmit data from individual vision tests of people with normalized vison for building a normative database, which may be used to improve accuracy or reduce test time of the vision tests conducted on users. Further, vision tests conducted by using the HMVD of the present specification, allow for archiving, storing and processing data from the tests as the test results are digitized, and the HMVD may be connected to other computing devices over a network.

In an embodiment a VFT test conducted via an HMVD may be tailored based on historical measurements, as an HMVD enables presentation of varied types of stimuli anywhere in a user's visual field thereby allowing for changes in the nature of the information obtained via the VFT. In an embodiment, the frequency of stimuli presented at the boundaries between areas where the user historically has poor contrast sensitivity and good contrast sensitivity, is increased, and the frequency of stimuli presented in the middle of regions where the user historically has very poor or very good vision is decreased by storing the user's historical data, translating areas of such sensitivity into pixel coordinates, and programming the stimuli generator to increase a frequency of presentation of stimuli in those pixel coordinates. The stimuli presented at the boundary points provide information for estimating a user's visual field.

In some embodiments, damage to a specific type of retinal ganglion cell (output cell of the retina) may be tested by presenting stimuli with a temporal profile that the retinal ganglion cell type is most responsive to. Among retinal ganglion cells, parvocellular cells (P cells) are more sensitive to a gradual ramp-up and ramp-down of stimuli (i.e., low temporal frequency) while magnocellular cells (M cells) are more sensitive to an abrupt onset and offset (i.e., high temporal frequency). Standard automated perimetry uses stimuli with square wave temporal profiles with an abrupt onset and offset (i.e., high temporal frequency), however, in embodiments, with the use of an HMVD the temporal profile of the stimulus can be changed to a Gaussian (i.e., low temporal frequency) that preferentially tests for damage among P cells instead of M cells.

In some embodiments, doctors/clinicians may use an HMVD to perform a rapid VFT that only presents stimuli at maximum contrast at each location a predefined number of times ranging from one to three times. This rapid screening test cannot be used to estimate contrast sensitivity at each test location, but it provides a binary map of regions with “holes” or “scotomas”. The use of a HMVD also enables presentations of stimulus in a random manner to the user's eyes, without the user knowing to which eye the stimulus is being presented. In some cases, the VFT may be focused on testing at just the edges of a user's scotoma, or the entire test may be run using prior test results in order to obtain a “normative database” specific to the user. In an embodiment, a doctor/clinician may present a custom set of test points during the test to obtain higher resolution data at certain locations in the user's visual field. In various embodiments, the use of an HMVD for conducting the VFT allows for greater efficiency, reduced time, individual testing of each eye, testing of specific areas within the visual field of each eye, and testing of specific cell types in the retina of each eye, as compared to conducting the test manually. In an embodiment, the VFT results may be used to configure an HMVD with a gravity lens filter, which provides the user with an image that is distorted around scotomas, but unlike most other filtering techniques retains all image content (i.e., no information in the image is lost).

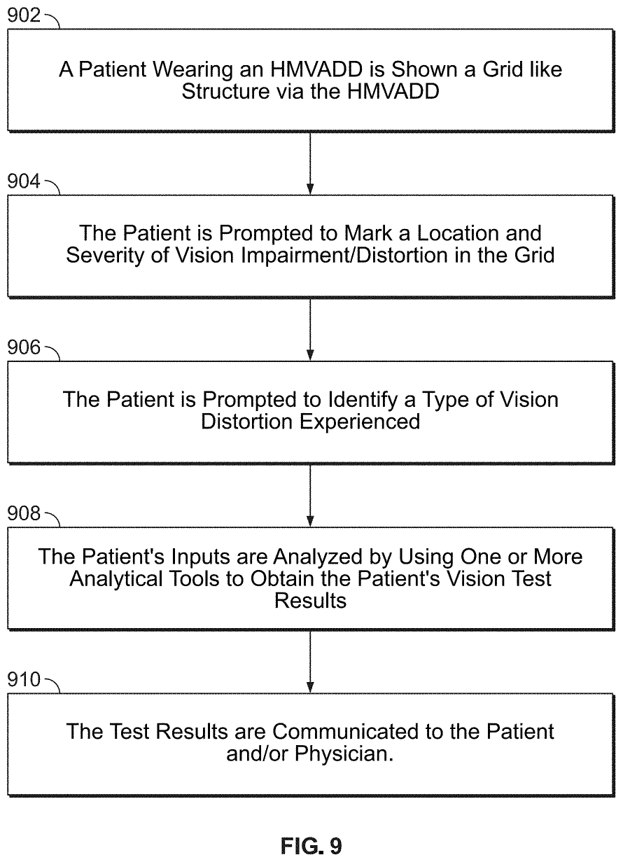

FIG. 9 is a flowchart illustrating the steps of conducting an Amsler grid test by using an HMVD, in accordance with an embodiment of the present specification. At step 902, a user wearing an HMVD is presented with a grid-like structure via the HMVD. In embodiments, the presented structures may be lines, checkerboard patterns or common objects with straight lines known by the user, such as, but not limited to a chair or a table. At step 904 the user is prompted to mark a location and severity of vision impairment/distortion in the grid. In embodiments, the user may mark a location in the grid by using voice commands and naming a grid coordinate, by using a joystick coupled with the HMVD, or by using gestures recognized by the HMVD via gyro sensors coupled with the HMVD. In an embodiment, the user may quantify a severity of vision impairment/distortion by using a number rating on a predefined scale presented via the HMVD. At step 906, the user is prompted to identify a type of vision distortion experienced. In an embodiment, the user may identify the type of vision distortion by using predefined keywords or by selecting a suitable response from a plurality of options presented via the HMVD. At step 908 the user's responses are analyzed by using one or more analytical tools to obtain the user's vision test results. At step 910 the test results are communicated to the user.

In embodiments the results of the vision tests may be improved by conducting said tests via the HMVD, as pre-recorded data about the user and/or corresponding health conditions of the user may be accessed. In an embodiment, the test parameters for a plurality of vision tests are stored and a comparison of the corresponding diagnoses is conducted to enable a doctor to retroactively understand which test was best suited to diagnose a user's visual condition. In an embodiment, the tests parameters for a plurality of vision tests are stored and compared with the tests conducted at a later date to enable a doctor to understand which test was best suited to predict future impairments of a user's visual condition.

In some embodiments, the system is configured to recommend one or more of a plurality of vision tests to the user based on a diagnosis or condition of the user. In some embodiments, the user's diagnosis or condition is provided as input to the system—such as, for example, through one or more GUIs accessible to the user on his smartphone. The system associates the recommended one or more of a plurality of vision tests with the user and schedules the tests for administration to the user. In some embodiments, the schedule of the one or more vision tests may be automatically incorporated in the user's calendar on his smartphone and corresponding prompts may be generated as reminders. In embodiments, the clinician is provided an option of keeping, modifying, deleting, or replacing any of the one or more of the plurality of vision tests.

In embodiments, predictive analytics is used with test parameters of a plurality of tests performed along with a sequence of performing said tests to predict a time series of developing a vision condition/abnormality for a user. In an embodiment, various analytics tools may be used to determine vision tests best suited for a given age, demographic, location, time of day, climate and HMD model. In an embodiment, analytics tools may be used to correlate measurement of a user's eye movement during a vision test as well as other test parameters mentioned above to predict the accuracy of the test being performed. For example, frequent or quick eye movement may be an indication of reduced test quality. In embodiments, analytic techniques such as, but not limited to, correlation and regression are used, with linear and polynomial regression being used for continuous variables, and logistic regression being used for categorical variables. In an embodiment, statistical significance may be calculated to show that a new data point is significantly different from previous ones. However, depending on the nature of the data, in different embodiments, many other types of data analysis may be performed.

In an embodiment, the Amsler grid test is conducted on a user without the use of a grid, wherein the test is conducted via an HMVD, and wherein the smartphone that used in the HMVD (as shown in FIGS. 1A, 1B and 1C) is not located in the headset. Instead, the smartphone may be used by the user as a pointer during the test, as the phone comprises a MEMS gyroscope. In an embodiment, the user is prompted to point to a location that will be the “origin” of a coordinate axis, the location being where the user's eyes are focused. After pointing, the user is prompted to press a predefined button on the phone, in order to mark the location of the origin. Thus, in embodiments, before commencing the test, it is required that the smart phone is placed right in front of the user's eyes and that the user press a predefined button on the phone to mark a location of the origin which has the co-ordinates (0,0,0) in the 3D space before the user's eyes.

In embodiments, a 3D coordinate system is predefined and communicated to the smartphone. In an embodiment, the phone is moved outwards and towards an imaginary fixation point (straight ahead) and a predefined button on the phone is pressed to mark the z-axis, after which, two more predefined buttons on the phone are pressed to mark the x- and y-axes respectively, by prompting the user to hold the phone up above his/her head for the y-axis and to the right of his/her eyes for the x-axis. In an alternate embodiment, the x, y and z axes are marked by just by the user rotating the phone and pointing the phone in the directions of the axes, in which case the user is not required to actually move his/her arms about. Thereafter, the user is prompted to look straight ahead and use the phone to draw/mark any regions in his/her visual field that the user cannot see and any regions marked by the user are analyzed to obtain the test result for the user, as described with respect to FIG. 9.

In some embodiments, the test result generated from the Amsler grid test conducted by using the above technique may be used to refine VFT test points. For example, in cases where the VFT is used for mapping out the scotomas in a user's visual field, the boundaries of Amsler grid regions drawn by the user may be used as test points in the VFT. Alternatively, in order to determine whether perceived distortions within a region affects a user's contrast sensitivity, points within the Amsler grid regions may be used as VFT test points. In embodiments, if the user reports no vision loss in certain regions through an Amsler grid test, there is less need to test the user's vision within those regions with a VFT.

Thus, data generated from a first test, such as the Amsler grid test, is used to modify the administration, processing or analysis of a second test, such as the VFT, subsequently or in future. For example, boundaries of scotomas, for a user, are of interest. If data from a test provides or points to one or more affected areas where the user cannot see, subsequent tests may concentrate test points in those one or more affected areas. In some embodiments, to minimize the amount of time, the system is configured to increase the amount of test points (that is, increase the density of test points in the identified or affected one or more areas) or frequency of the stimulus in those one or more affected areas. Accordingly, in some embodiments, the clinician may provide input indicative of the areas where to test more or the areas may be identified automatically by the system based on at least one prior conducted vision test. Preferably the system would present the Amsler grid first (since it is faster). VFT gives you more data but not as fast. While any of the vision tests may be conducted first, in some embodiments it is preferred to perform the Amsler grid test first (then followed by the VFT) since it is faster.

As another example, data from the first and second tests is compared and correlated to assess reliability of the tests. If the system determines that there is a misalignment between the results of one test and the results of another test, that may indicate unreliable results. If a first test yielded a vision assessment that is better than the second test, then it may mean the first or second test is unreliable, since that kind of improvement may be unlikely. In general, unexpected or unlikely test data contradictions are indicative of unreliability. Accordingly, the system is configured to generate a notification and report for the clinician in case of identified contradictions or unreliability in test data.

In various embodiments, when the Amsler grid test is conducted without using a gird, as above, it is preferred that it occurs in an environment with a predefined minimum number of features so that the user can see distortions in the presented features. In embodiments, where the user is not able to identify/mark the areas in his/her visual field where the user's vision is impaired, the use of a grid becomes imperative for conducting the Amsler grid test, as explained above.

As is known, VFT and Amsler grid vision tests provide different types of information, as the VFT is used to estimate sensitivity (i.e., probability of detecting a stimulus) across the visual field. In an embodiment, the HMVD is used to combine the results of the two tests on a display for analysis by a doctor. In an embodiment, the output of each test conducted by using a HMVD is combined on a display so that a doctor can see which parts of a user's visual field exhibit less sensitivity (determined via the VFT) in relation to the parts exhibiting visual disturbances caused by changes in the user's retina (obtained via the Amsler grid test).

An Amsler grid test may be used to provide different types of information regarding a user's vision, depending upon the manner in which the test is constructed. Hence by asking a wide variety of questions from the user, a variety of information may be obtained. In an exemplary scenario a user may be asked to only point out regions in the user's visual field within which lines are distorted or blurry, but not where there is no “hole” or a “dark area”. Conversely, in other embodiments, the user may be asked to point out regions with a “hole” or “dark area” rather than perceiving distortions. In the first case, there may or may not be loss of contrast sensitivity, while in the second case there will be little to no loss of contrast sensitivity.

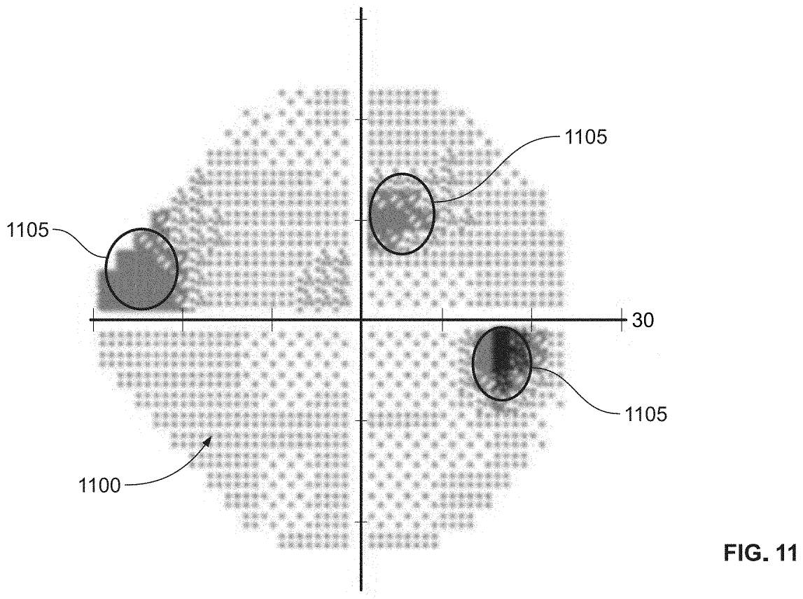

In combining the display of results from both vision tests a doctor may easily interpret the test results and see changes in a user's visual field frame-by-frame as a function of time. In an embodiment, aggregating and/or combining the test results is performed by overlaying the Amsler grid region results on the output of the VFT. FIG. 11 illustrates a grayscale map 1100 corresponding to a VFT test data. The areas identified with circles 1105 are indicative of the Amsler grid data overlaid on the VFT test data. Further, in embodiments, the combined measurements from both the vision tests may be displayed in a graphical display of affected visual field areas, showing interdependencies between time, eye conditions and other test parameters.

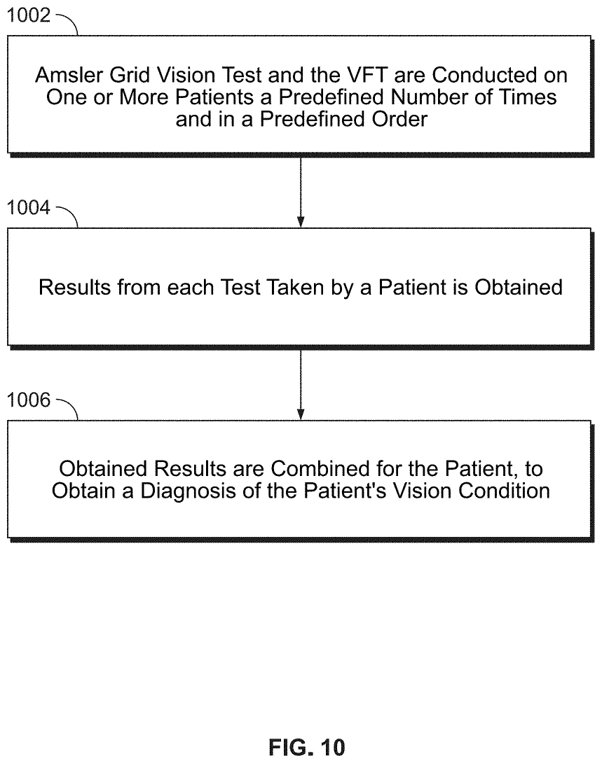

FIG. 10 is a flowchart illustrating the steps of conducting a combination of Amsler grid test and VFT by using an HMVD, in accordance with an embodiment of the present specification. At step 1002 the Amsler grid vision test and the VFT are conducted on one or more users a predefined number of times and in a predefined order. In an embodiment, each user is made to take the two tests alternately and repeatedly at different times for measuring the user's vision impairment. In an embodiment, different users grouped in accordance with parameters such as, but not limited to, their demographics, location, treating physician, eye condition or treatment protocol being followed, are made to take the tests together. At step 1004 the results from each test taken by a user is obtained. At step 1006 the obtained results are combined for the user, to obtain a diagnosis of the user's vision condition.

In embodiments, the results of the two tests are combined to obtain an aggregate measurement that includes a superimposed graphic overlay, showing interdependencies between timing of test, eye conditions and user groups. In various embodiments, such combination of results enables improvement in test accuracy, and identification of similarities and differences between user groups along with providing information about the progression of a user's eye condition.

In embodiments, the Amsler grid and VFT tests conducted via an HMVD may be varied in order to improve diagnostics accuracy and localization of areas with vision impairment, depending on type of visual distortion. In an embodiment, the tests may be randomized by varying test parameters such as but not limited to: degree of visual angle, i.e. size as observed by user; color, line thickness and resolution; ambient light; presented stimulus, such that the identification allows for testing of acuity in peripheral, e.g. Landolt C; location of stimulus by presenting movement in order to test motion perception in peripheral vision; fixation targets, including presenting stimulus in blind spot that should not be visible, and which implies that a user's eye is likely not fixated on the target, which is typically located in the center; eye that is presented the stimulus (left or right).

In embodiments, eye tracking is used to present the test stimulus to a user via the HMVD, which also enables checking if the user is looking at a required center. In embodiments the combined test results may be used to configure a HMVD with a filter that compensates for the identified visual impairments.

Further, the combined test results may be used to adjust HMVD parameters such as screen brightness, resolution, distance from the eyes, lens, gamma, ambient light and other parameters of the HMVD lens that need to be tuned for each device. In an embodiment, the vision test results are evaluated to optimize the test, by comparing test configurations to a user's final diagnosis that may be derived from different tests or tests performed at different times to identify the most suitable configurations of parameters mentioned above.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of evaluating a user's visual field using a head-mounted device configured to be positioned on the user's head, wherein the device comprises at least one processor, a display in data communication with the at least one processor, and a non-transient memory in data communication with the at least one processor and adapted to store programmatic instructions that, when executed, execute said method, the method comprising:
generating a first plurality of visual stimuli, wherein the first plurality of visual stimuli is presented in a form of a grid defined by two or more vertical lines intersecting two or more horizontal lines, wherein the grid covers a first plurality of coordinate locations in the visual field, and wherein each of the first plurality of visual stimuli has at least one of a first plurality of characteristics;
causing the first plurality of visual stimuli to be displayed on the display in accordance with its first plurality of characteristics;
detecting a discrepancy based on a comparison of the first plurality of characteristics with a user's response that is indicative of the visual characteristics of the first plurality of visual stimuli experienced by the user;
storing the detected discrepancy as a first set of data;
using the first set of data to generate a second plurality of visual stimuli, wherein each of the second plurality of visual stimuli has at least one of a second plurality of characteristics and is associated with a second plurality of coordinate locations and wherein a number of the second plurality of coordinate locations is less than a number of the first plurality of coordinate locations;
causing each of the second plurality of visual stimuli to be displayed on said display in accordance with its one of the second plurality of coordinate locations and one of the second plurality of characteristics;
receiving responses from the user, wherein the responses are indicative of the visual characteristics of the second plurality of visual stimuli experienced by the user; and
determining attributes of the user's visual field based on the detected discrepancy and the responses that are indicative of the visual characteristics of the second plurality of visual stimuli experienced by the user.

2. The method of claim 1, wherein the discrepancy is indicative of one or more deficits in the visual field and wherein the discrepancy is at least one of a partially missing vertical line, a partially missing horizontal line, a partially wavy vertical line, a partially wavy horizontal line, a partially blurred vertical line, or a partially blurred horizontal line.

3. The method of claim 1, further comprising associating a first coordinate location from the first plurality of coordinate locations with the discrepancy and storing the detected discrepancy and first coordinate location as the first set of data.

4. The method of claim 3, wherein the second plurality of coordinate locations are only positioned at the first coordinate location.

5. The method of claim 1, wherein the grid covers an entirety of the visual field of the user.

6. The method of claim 1, wherein the grid is an Amsler grid.

7. The method of claim 1, wherein the grid is defined by at least five vertical lines intersecting at least five horizontal lines to create equally sized boxes.

8. The method of claim 1, wherein detecting the discrepancy is further achieved by a) receiving the response from the user, wherein the response is indicative of the visual characteristics of the first plurality of visual stimuli experienced by the user and b) comparing the visual characteristics of the first plurality of visual stimuli experienced by the user with the first plurality of characteristics to identify the discrepancy.

9. The method of claim 1, wherein at least one of the second plurality of coordinate locations, a temporal frequency of the second plurality of visual stimuli, or the second plurality of characteristics of the second plurality of visual stimuli presented to a left eye of the user differs from at least one of the second plurality of coordinate locations, a temporal frequency of the second plurality of visual stimuli, or the second plurality of characteristics of the second plurality of visual stimuli presented to a right eye of the user.

10. The method of claim 1, further comprising using the first set of data to determine a first area in the visual field having a determined detection below a predefined value and a second area in the visual field having a determined detection above a predefined value.

11. The method of claim 10, further comprising setting a temporal frequency of the second plurality of visual stimuli in the first area different from a temporal frequency of the second plurality of visual stimuli in the second area.

12. The method of claim 1, further comprising using the first set of data to determine a region that is smaller than the visual field of the user corresponding to an area of visual impairment.

13. The method of claim 12, further comprising presenting the second plurality of visual stimuli only within said region.

14. The method of claim 1, further comprising using the determined attributes to identify one or more regions of visual impairment in the visual field.

15. The method of claim 14, further comprising generating a display wherein the display visually overlays the one or more regions onto the visual field.

16. A computer program product for evaluating a user's visual field and configured to be executed in a head-mounted device configured to be positioned on the user's head, wherein the device comprises at least one processor, a display in data communication with the at least one processor, and a non-transient memory in data communication with the at least one processor and adapted to store the computer program product, wherein, when executed, the computer program product is configured to evaluate the user's visual field by:
generating a first plurality of visual stimuli, wherein the first plurality of visual stimuli is presented in a form of a grid defined by two or more vertical lines intersecting two or more horizontal lines, wherein the grid covers a first plurality of coordinate locations in the visual field, and wherein each of the first plurality of visual stimuli has at least one of a first plurality of characteristics;
causing the first plurality of visual stimuli to be displayed on the display in accordance with its first plurality of characteristics;
detecting a discrepancy based on a comparison of the first plurality of characteristics with a user's response that is indicative of the visual characteristics of the first plurality of visual stimuli experienced by the user;
storing the detected discrepancy as a first set of data;
using the first set of data to generate a second plurality of visual stimuli, wherein each of the second plurality of visual stimuli has at least one of a second plurality of characteristics and is associated with a second plurality of coordinate locations and wherein a number of the second plurality of coordinate locations is less than a number of the first plurality of coordinate locations;
causing each of the second plurality of visual stimuli to be displayed on said display in accordance with its one of the second plurality of coordinate locations and one of the second plurality of characteristics;
receiving responses from the user, wherein the responses are indicative of the visual characteristics of the second plurality of visual stimuli experienced by the user; and
determining attributes of the user's visual field based on the detected discrepancy and the responses that are indicative of the visual characteristics of the second plurality of visual stimuli experienced by the user.

17. The computer program product of claim 16, wherein the discrepancy is indicative of one or more deficits in the visual field and wherein the discrepancy is at least one of a partially missing vertical line, a partially missing horizontal line, a partially wavy vertical line, a partially wavy horizontal line, a partially blurred vertical line, or a partially blurred horizontal line.

18. The computer program product of claim 16, further configured to associate a first coordinate location from the first plurality of coordinate locations with the discrepancy and store the detected discrepancy and first coordinate location as the first set of data.

19. The computer program product of claim 18, wherein the second plurality of coordinate locations are only positioned at the first coordinate location.

20. The computer program product of claim 16, wherein the grid covers an entirety of the visual field of the user.

21. The computer program product of claim 16, wherein the grid is an Amsler grid.

22. The computer program product of claim 16, wherein the grid is defined by at least five vertical lines intersecting at least five horizontal lines to create equally sized boxes.

23. The computer program product of claim 16, wherein detecting the discrepancy is further achieved by a) receiving the response from the user, wherein the response is indicative of the visual characteristics of the first plurality of visual stimuli experienced by the user and b) comparing the visual characteristics of the first plurality of visual stimuli experienced by the user with the first plurality of characteristics to identify the discrepancy.

24. The computer program product of claim 16, wherein at least one of the second plurality of coordinate locations, a temporal frequency of the second plurality of visual stimuli, or the second plurality of characteristics of the second plurality of visual stimuli presented to a left eye of the user differs from at least one of the second plurality of coordinate locations, a temporal frequency of the second plurality of visual stimuli, or the second plurality of characteristics of the second plurality of visual stimuli presented to a right eye of the user.

25. The computer program product of claim 16, further configured to use the first set of data to determine a first area in the visual field having a determined detection below a predefined value and a second area in the visual field having a determined detection above a predefined value.

26. The computer program product of claim 25, further configured to set a temporal frequency of the second plurality of visual stimuli in the first area different from a temporal frequency of the second plurality of visual stimuli in the second area.

27. The computer program product of claim 16, further configured to use the first set of data to determine a region that is smaller than the visual field of the user corresponding to an area of visual impairment.

28. The computer program product of claim 27, further configured to present the second plurality of visual stimuli only within said region.

29. The computer program product of claim 16, further configured to use the determined attributes to identify one or more regions of visual impairment in the visual field.

30. The computer program product of claim 29, further configured to generate a display wherein the display visually overlays the one or more regions onto the visual field.
